(19) United States
(12) Patent Application Publication
(10) Pub. No.: US 2013/0090333 A1
(43) Pub. Date: Apr. 11, 2013

(54) BENZOXAZINE DERIVATIVES AS CRAC MODULATORS

(71) Applicant: Hoffmann-La Roche Inc., Nutley, NJ (US)

(72) Inventors: Niala Bhagirath, Bloomfield, NJ (US); Kenneth Albert Brameld, Menlo Park, CA (US); Joshua Kennedy-Smith, New York, NY (US)

(73) Assignee: Hoffmann-La Roche Inc., Nutley, NJ (US)

(21) Appl. No.: 13/626,117

(22) Filed: Sep. 25, 2012

Related U.S. Application Data
(60) Provisional application No. 61/543,436, filed on Oct. 5, 2011.

(51) Int. Cl. A61K 31/538; C07D 413/10; A61P 11/06; C07D 417/14; A61P 19/02; A61P 11/00; C07D 265/36; C07D 413/14

(52) U.S. Cl. 514/230.5; 544/105

(57) ABSTRACT

Compounds of the formula (I):

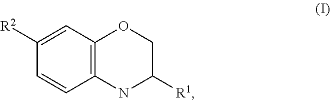

or pharmaceutically acceptable salts thereof, wherein R¹ and R² are as defined herein. Also disclosed are methods of making the compounds and using the compounds for treatment of diseases associated with calcium release-activated calcium channels (CRAC).

### PRIORITY TO RELATED APPLICATION(S)

This application claims the benefit of U.S. Provisional Application No. 61/543,436, filed Oct. 5, 2011, which is hereby incorporated by reference in its entirety.

### FIELD OF THE INVENTION

This invention pertains to compounds useful for treatment of autoimmune and inflammatory diseases associated with IL-2 inhibition via modulation of calcium release-activated calcium channels.

### BACKGROUND OF THE INVENTION

The cytokine interleukin 2 (IL-2) is a T-cell mitogen important for T-cell proliferation and as a B cell growth factor. Because of its effects on T cells and B cells, IL-2 is recognized as an important regulator of immune responses. IL-2 is involved in inflammation, tumor progression and hematopoiesis, and IL-2 affects the production of other cytokines such as TNA alpha, TNF beta, IFN gamma. Inhibition of IL-2 production thus is relevant to immunosuppression therapies and treatment of inflammatory and immune disorders.

T-cell antigen binding in inflammatory events leads to T-cell initiated calcium influx by calcium release-activated calcium channels (CRAC). IL-2 secretion by T-cells occurs in response to calcium ion influx. Modulation of CRAC thus provides a mechanism for control of production of IL-2 and other cytokines associated with inflammation. CRAC inhibition has been recognized as a potential route to therapies for rheumatoid arthritis, asthma, allergic reactions and other inflammatory conditions (see, e.g., Chang et al., Acta Pharmacologica Sinica (2006) Vol. 7, 813-820), and CRAC inhibitors have been shown to prevent antigen-induced airway eosinophilia and late phase asthmatic responses via Th2 cytokine inhibition in animal models (Yoshino et al., Eur. J. Pharm. (2007) Vol. 560(2), 225-233). There is, accordingly, a need for CRAC inhibitors.

### SUMMARY OF THE INVENTION

The invention provides compounds of the formula (I):

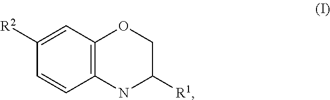

wherein:
R¹ is phenyl, unsubstituted or mono- or bi-substituted independently with halogen; and

### R² is:

phenyl, unsubstituted or mono- or bi-substituted independently with lower alkyl, halogen, halo-lower alkyl, alkoxy, unsubstituted five-membered heteroaryl ring or five-membered heteroaryl ring substituted with lower alkyl;

pyridine, unsubstituted or mono- or bi-substituted independently with lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety; or

a five-membered heteroaryl ring, unsubstituted or mono- or bi-substituted independently with lower alkyl, halogen, halo-lower alkyl, alkoxy, unsubstituted five-membered heteroaryl ring, five-membered heteroaryl ring substituted with lower alkyl, unsubstituted six-membered heteroaryl ring or six-membered heteroaryl ring substituted with lower alkyl;

or a pharmaceutically acceptable salt thereof.

The invention also provides for pharmaceutical compositions comprising the compounds, methods of using the compounds, and methods of preparing the compounds.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Amino” means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Heteroaryl” means a monocyclic or bicyclic radical of 5 to 12 ring atoms having at least one aromatic ring containing one, two, three or four ring heteroatoms selected from N, O, or S, the remaining ring atoms being C. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, tetrazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

The terms “halo”, “halogen” and “halide”, which may be used interchangeably, refer to a substituent fluoro, chloro, bromo, or iodo.

“Haloalkyl” and “halo-lower alkyl” means alkyl and lower alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or
salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes: preventing the disease state, i.e. causing the clinical symptoms of the disease state not to develop in a subject that may be exposed to or predisposed to the disease state, but does not yet experience or display symptoms of the disease state; inhibiting the disease state, i.e., arresting the development of the disease state or its clinical symptoms; or relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structures herein are intended to encompass all naturally occurring isotopes of such atoms. Thus, for example, the hydrogen atoms represented herein are meant to include deuterium and tritium, and the carbon atoms are meant to include C¹³ and C¹⁴ isotopes.

All patents and publications identified herein are incorporated herein by reference in their entirety.

The invention provides compounds of the formula I:

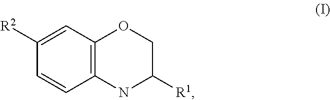

wherein:
R¹ is phenyl, unsubstituted or mono- or bi-substituted independently with halogen; and

### R² is:

phenyl, unsubstituted or mono- or bi-substituted independently with lower alkyl, halogen, halo-lower alkyl, alkoxy, unsubstituted five-membered heteroaryl ring or five-membered heteroaryl ring substituted with lower alkyl;

pyridine, unsubstituted or mono- or bi-substituted independently with lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety; or

a five-membered heteroaryl ring, unsubstituted or mono- or bi-substituted independently with lower alkyl, halogen, halo-lower alkyl, alkoxy, unsubstituted five-membered heteroaryl ring, five-membered heteroaryl ring substituted with lower alkyl, unsubstituted six-membered heteroaryl ring or six-membered heteroaryl ring substituted with lower alkyl;

or a pharmaceutically acceptable salt thereof.

In another embodiment of the invention, provided is a compound according to formula (I), wherein R¹ is phenyl mono- or bi-substituted independently with F or Cl.

In another embodiment of the invention, provided is a compound according to formula (I), wherein R¹ is difluoro-phenyl.

In another embodiment of the invention, provided is a compound according to formula (I), wherein R¹ is chloro-fluoro-phenyl.

In another embodiment of the invention, provided is a compound according to formula (I), wherein R² is phenyl, unsubstituted or mono- or bi-substituted independently with lower alkyl, halogen, halo-lower alkyl, alkoxy, unsubstituted five-membered heteroaryl ring or five-membered heteroaryl ring substituted with lower alkyl.

In another embodiment of the invention, provided is a compound according to formula (I), wherein R² is phenyl substituted with —CF₃ and —CH₃.

In another embodiment of the invention, provided is a compound according to formula (I), wherein R² is pyridine, unsubstituted or mono- or bi-substituted independently with lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety.

In another embodiment of the invention, provided is a compound according to formula (I), wherein R² is pyridine, unsubstituted or mono- or bi-substituted independently with —CH₃, —OCH₃, —SO₂CH₂CH₃, chlorine, oxazole, methyl-pyrimidine-amine, methyl-thiazole or methyl-tetrazole.

In another embodiment of the invention, provided is a compound according to formula (I), wherein R² is a five-membered heteroaryl ring, unsubstituted or mono- or bi-substituted independently with lower alkyl, halogen, halo-lower alkyl, alkoxy, unsubstituted five-membered heteroaryl ring, five-membered heteroaryl ring substituted with lower alkyl, unsubstituted six-membered heteroaryl ring or six-membered heteroaryl ring substituted with lower alkyl.

In another embodiment of the invention, provided is a compound according to formula (I), wherein R² is pyrazole, triazole or thiazole, mono- or bi-substituted independently with —CF3, methyl, ethyl, pyridine, pyrazine methyl-pyridine, oxazole or methyl-oxazole.

In another embodiment of the invention, provided is a compound according to formula (I) wherein the compound is:
3-(2,6-Difluoro-phenyl)-7-(2-methyl-4-trifluoromethyl-phenyl)-3,4-dihydro-2H-benzo[1,4]oxazine;
(R)-3-(2,6-Difluoro-phenyl)-7-(2-methyl-4-trifluoromethyl-phenyl)-3,4-dihydro-2H-benzo[1,4]oxazine or (S)-3-(2,6-Difluoro-phenyl)-7-(2-methyl-4-trifluoromethyl-phenyl)-3,4-dihydro-2H-benzo[1,4]oxazine;
(S)-3-(2,6-Difluoro-phenyl)-7-(2-methyl-4-trifluoromethyl-phenyl)-3,4-dihydro-2H-benzo[1,4]oxazine or (R)-3-(2,6-Difluoro-phenyl)-7-(2-methyl-4-trifluoromethyl-phenyl)-3,4-dihydro-2H-benzo[1,4]oxazine;
3-(2,6-difluoro-phenyl)-7-(2-methyl-5-trifluoromethyl-phenyl)-3,4-dihydro-2H-benzo[1,4]oxazine;
3-(2,6-Difluoro-phenyl)-7-(2-methyl-5-trifluoromethyl-2H-pyrazol-3-yl)-3,4-dihydro-2H-benzo[1,4]oxazine;
3-(2,6-Difluoro-phenyl)-7-(2-ethyl-5-trifluoromethyl-2H-pyrazol-3-yl)-3,4-dihydro-2H-benzo[1,4]oxazine;
3-(2,6-Difluoro-phenyl)-7-(2-ethyl-5-pyridin-3-yl-2H-pyrazol-3-yl)-3,4-dihydro-2H benzo[1,4]oxazine;
3-(2,6-Difluoro-phenyl)-7-(2-ethyl-5-pyrazin-2-yl-2H-pyrazol-3-yl)-3,4-dihydro-2H-benzo[1,4]oxazine;
3-(2,6-Difluoro-phenyl)-7-(2-methyl-5-pyridin-3-yl-2H-[1,2,4]triazol-3-yl)-3,4-dihydro-2H-benzo[1,4]oxazine;
3-(2,6-Difluoro-phenyl)-7-(2-ethyl-5-pyridin-3-yl-2H-[1,2,4]triazol-3-yl)-3,4-dihydro-2H-benzo[1,4]oxazine;
3-(2,6-Difluoro-phenyl)-7-(5-methyl-2-pyridin-2-yl-thiazol-4-yl)-3,4-dihydro-2H-benzo[1,4]oxazine;
3-(2,6-Difluoro-phenyl)-7-(5-methyl-2-pyridin-3-yl-thiazol-4-yl)-3,4-dihydro-2H-benzo[1,4]oxazine;
3-(2,6-Difluoro-phenyl)-7-(5-ethyl-2-pyridin-2-yl-thiazol-4-yl)-3,4-dihydro-2H-benzo[1,4]oxazine;
3-(2,6-Difluoro-phenyl)-7-(5-ethyl-2-pyridin-3-yl-thiazol-4-yl)-3,4-dihydro-2H-benzo[1,4]oxazine;
3-(2,6-difluorophenyl)-7-(5-methyl-2-(pyrazin-2-yl)thiazol-4-yl)-3,4-dihydro-2H-benzo[b][1,4]oxazine;
3-(2,6-difluorophenyl)-7-(5-ethyl-2-(pyrazin-2-yl)thiazol-4-yl)-3,4-dihydro-2H-benzo[b][1,4]oxazine;
3-(2,6-Difluoro-phenyl)-7-(5-ethyl-2-pyridin-4-yl-thiazol-4-yl)-3,4-dihydro-2H-benzo[1,4]oxazine;
3-(2,6-Difluoro-phenyl)-7-(5-ethyl-2-(2-ethyl-pyridin-4-yl)-thiazol-4-yl)-3,4-dihydro-2H-benzo[1,4]oxazine;
3-(2,6-Difluoro-phenyl)-7-(5-ethyl-2-oxazol-2-yl-thiazol-4-yl)-3,4-dihydro-2H-benzo[1,4]oxazine;
3-(2,6-difluorophenyl)-7-(5-ethyl-2-(5-methylisoxazol-3-yl)thiazol-4-yl)-3,4-dihydro-2H-benzo[b][1,4]oxazine;
3-(2,6-Difluoro-phenyl)-7-(6-methoxy-4-methyl-pyridin-3-yl)-3,4-dihydro-2H-benzo[1,4]oxazine;
3-(2,6-difluorophenyl)-7-(6-(ethylsulfonyl)-4-methylpyridin-3-yl)-3,4-dihydro-2H-benzo[b][1,4]oxazine;
7-(6-Chloro-4-methyl-pyridin-3-yl)-3-(2,6-difluoro-phenyl)-3,4-dihydro-2H-benzo[1,4]oxazine;
3-(2,6-Difluoro-phenyl)-7-(4-methyl-6-oxazol-2-yl-pyridin-3-yl)-3,4-dihydro-2H-benzo[1,4]oxazine;
5-(5-(3-(2,6-difluorophenyl)-3,4-dihydro-2H-benzo[b][1,4]oxazin-7-yl)-4-methylpyridin-2-yl)pyrimidin-2-amine;
3-(2,6-difluorophenyl)-7-(4-methyl-6-(2-methylthiazol-5-yl)pyridin-3-yl)-3,4-dihydro-2H-benzo[b][1,4]oxazine;
3-(2,6-difluorophenyl)-7-(4-methyl-6-(2-methyl-2H-tetrazol-5-yl)pyridin-3-yl)-3,4-dihydro-2H-benzo[b][1,4]oxazine;
3-(2,6-Difluoro-phenyl)-7-(2-methyl-5-oxazol-2-yl-phenyl)-3,4-dihydro-2H-benzo[1,4]oxazine;
3-(2-Chloro-6-fluoro-phenyl)-7-(2-methyl-4-trifluoromethyl-phenyl)-3,4-dihydro-2H-benzo[1,4]oxazine;
3-(2-Chloro-6-fluoro-phenyl)-7-(2-methyl-5-trifluoromethyl-phenyl)-3,4-dihydro-2H-benzo[1,4]oxazine;
3-(2-Chloro-6-fluoro-phenyl)-7-(5-methyl-2-pyridin-2-yl-thiazol-4-yl)-3,4-dihydro-2H-benzo[1,4]oxazine;
3-(2-Chloro-6-fluoro-phenyl)-7-(5-methyl-2-pyridin-3-yl-thiazol-4-yl)-3,4-dihydro-2H-benzo[1,4]oxazine;
3-(2-Chloro-6-fluoro-phenyl)-7-(6-methoxy-4-methyl-pyridin-3-yl)-3,4-dihydro-2H-benzo[1,4]oxazine;
3-(2-chlorophenyl)-7-(1-methyl-3-(trifluoromethyl)-1H-pyrazol-5-yl)-3,4-dihydro-2H-benzo[b][1,4]oxazine;
3-(2-chlorophenyl)-7-(6-methoxy-4-methylpyridin-3-yl)-3,4-dihydro-2H-benzo[b][1,4]oxazine;
3-(2-chlorophenyl)-7-(5-methyl-2-(pyridin-3-yl)thiazol-4-yl)-3,4-dihydro-2H-benzo[b][1,4]oxazine or
3-(2-chlorophenyl)-7-(5-ethyl-2-(pyrazin-2-yl)thiazol-4-yl)-3,4-dihydro-2H-benzo[b][1,4]oxazine.

In another embodiment of the invention, provided is a compound according to formula (I) for use as a therapeutically active substance.

In another embodiment of the invention, provided is a pharmaceutical composition, comprising a therapeutically effective amount of a compound according to formula (I) and a therapeutically inert carrier.

In another embodiment of the invention, provided is the use of a compound according to formula (I) for the treatment or prophylaxis of arthritis or a respiratory disorder.

In another embodiment of the invention, provided is the use of a compound according to formula (I) for the preparation of a medicament for the treatment or prophylaxis of arthritis or a respiratory disorder.

In another embodiment of the invention, provided is a compound according to formula (I) for the treatment or prophylaxis of arthritis or a respiratory disorder.

In another embodiment of the invention, provided is a method for the treatment or prophylaxis of arthritis or a respiratory disorder, which method comprises the step of administering a therapeutically effective amount of a compound according to formula (I) to a patient in need thereof.

In another embodiment of the invention, provided is a method for the treatment or prophylaxis of arthritis or a respiratory disorder, wherein the respiratory disorder is chronic obstructive pulmonary disorder (COPD), asthma or bronchospasm.

In another embodiment, provided is the invention as hereinbefore described.

The invention also provides methods for treating a disease or condition mediated by or otherwise associated with a CRAC receptor, the method comprising administering to a subject in need thereof an effective amount of a compound of the invention.

The invention also provides methods for treating an inflammatory, respiratory or diabetes condition, the method comprising administering to a subject in need thereof an effective amount of a compound of the invention together with an effective amount of a CRAC inhibitor.

The disease may be an inflammatory disease such as arthritis, and more particularly rheumatoid arthritis, osteoarthritis, psoriasis, allergic dermatitis, asthma, chronic obstructive pulmonary disease, airways hyper-responsiveness, septic shock, glomerulonephritis, irritable bowel disease, and Crohn's disease.

The disease may be a pain condition, such as inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

The disease may be a respiratory disorder, such as chronic obstructive pulmonary disorder (COPD), asthma, or bronchospasm, or a gastrointestinal (GI) disorder such as Irritable Bowel Syndrome (IBS), Inflammatory Bowel Disease (IBD), biliary colic and other biliary disorders, renal colic, diarrhea-dominant IBS, pain associated with GI distension.

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

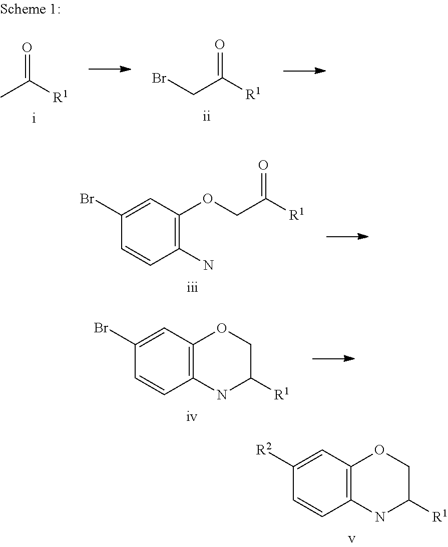

As shown in Scheme 1, acetophenone i can be converted to bromide ii, which can then be reacted with 4-bromo-2-hydroxy-aniline to give phenoxy acetophenone iii. Reductive cyclization of iii then gives 2-aryl-6-bromobenzoxazine iv. Suzuki coupling of iv with an appropriate boronic acid or ester then gives 2,6-diarylbenzoxazine v. As shown in Scheme 1, R¹ can be an aryl group, such as phenyl, unsubstituted or mono- or bi-substituted independently with, for example, halogen. R² can be an aryl group, such as phenyl, which can be unsubstituted or mono- or bi-substituted independently with, for example, lower alkyl, halogen, halo-lower alkyl, alkoxy, unsubstituted five-membered heteroaryl ring or five-membered heteroaryl ring substituted with lower alkyl. R² can also be a six-membered heteroaryl group, such as pyridine, unsubstituted or mono- or bi-substituted independently with, for example, lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety. R² can further be a five-membered heteroaryl ring, unsubstituted or mono- or bi-substituted independently with, for example, lower alkyl, halogen, halo-lower alkyl, alkoxy, unsubstituted five-membered heteroaryl ring, heteroaryl substituted with lower alkyl, unsubstituted six-membered heteroaryl ring or six-membered heteroaryl ring substituted with lower alkyl.

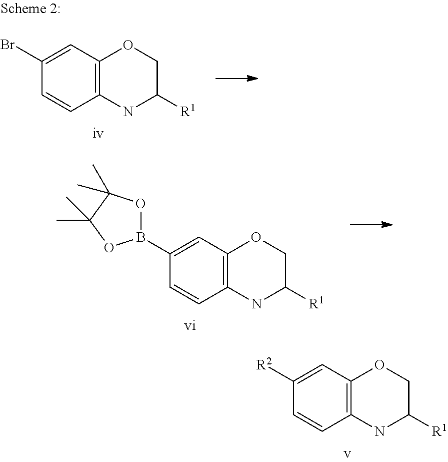

As shown in Scheme 2,2-aryl-6-bromobenzoxazine iv from Scheme 1 can also be converted to boronic ester vi. Suzuki coupling with aryl halides or triflates then provides access to 2,6-diarylbenzoxazine v. As shown in Scheme 2, R¹ can be an aryl group, such as phenyl, unsubstituted or mono- or bi-substituted independently with, for example, halogen. R² can be an aryl group, such as phenyl, which can be unsubstituted or mono- or bi-substituted independently with, for example, lower alkyl, halogen, halo-lower alkyl, alkoxy, unsubstituted five-membered heteroaryl ring or five-membered heteroaryl ring substituted with lower alkyl. R² can also be a six-membered heteroaryl group, such as pyridine, unsubstituted or mono- or bi-substituted independently with, for example, lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety. R² can further be a five-membered heteroaryl ring, unsubstituted or mono- or bi-substituted independently with, for example, lower alkyl, halogen, halo-lower alkyl, alkoxy, unsubstituted five-membered heteroaryl ring, heteroaryl substituted with lower alkyl, unsubstituted six-membered heteroaryl ring or six-membered heteroaryl ring substituted with lower alkyl.

Many variations on the procedure of the above Schemes are possible and will suggest themselves to those skilled in the art. Specific details for producing compounds of the invention are described in the Examples section below.

### Utility

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

Further, compounds of the invention are useful for treating respiratory disorders, including chronic obstructive pulmonary disorder (COPD), asthma, bronchospasm, and the like.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

### EXAMPLES

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Part I: Preparation of Certain Intermediates

### Intermediate 1

### Trifluoro-methanesulfonic acid 2-ethyl-5-trifluoromethyl-2H-pyrazol-3-yl ester

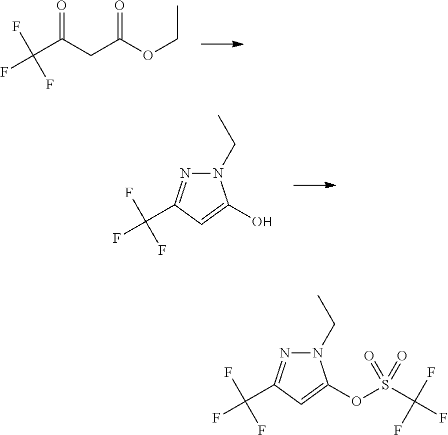 [SMILES: CCOC(=O)CC(=O)C(F)(F)F ; CCn1nc(C(F)(F)F)cc1O ; CCn1nc(C(F)(F)F)cc1OS(=O)(=O)C(F)(F)F]

ethyl-3-(trifluoromethyl)-1H-pyrazol-5(4H)-one: A mixture of ethyl 4,4,4-trifluoroacetoacetate (11.0 g, 59.7 mmol) and ethyl hydrazine oxalate (8.96 g, 59.7 mmol) in acetic acid (60 ml) was heated at 120° C. in a microwave reactor for 1.5 h. After irradiation the reaction mixture was poured into ice water, extracted with EtOAc. The organic phase was then washed with brine, dried over Na₂SO₄, filtered, concentrated under reduced pressure, and the crude material purified by flash chromatography (5-10% EtOAc/hexanes) to give 2-Ethyl-5-trifluoromethyl-2H-pyrazol-3-ol (4.62 g, 43%) as a yellow solid.

ethyl-3-(trifluoromethyl)-1H-pyrazol-5-yl trifluoromethanesulfonate: To a solution of 2-Ethyl-5-trifluoromethyl-2H-pyrazol-3-ol (4.41 g, 24.5 mmol) in CH₂Cl₂ (100 ml) and DIPEA (4.75 g, 36.7 mmol) at 0° C. was added trifluoromethane sulfonic anhydride (8.98 g, 31.8 mmol) dropwise. The mixture was stirred at 0° C. for 1 hour, then a cold solution of aqueous ammonium chloride and dichloromethane was added. The mixture was partitioned, and the organic phase washed with brine, dried over Na₂SO₄, filtered, concentrated under reduced pressure, and the crude material purified by filtering through a pad of silica (8% EtOAc/Hexanes) to give 1-ethyl-3-(trifluoromethyl)-1H-pyrazol-5-yl trifluoromethanesulfonate (6.12 g, 80%) as a yellow oil.

### Intermediate 2

### Trifluoro-methanesulfonic acid 2-ethyl-5-pyridin-3-yl-2H-pyrazol-3-yl ester

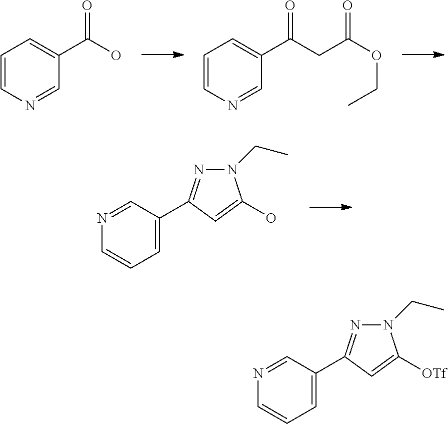 [SMILES: O=C(O)c1cccnc1 ; CCOC(=O)CC(=O)c1cccnc1 ; CCn1nc(-c2cccnc2)cc1O ; CCn1nc(-c2cccnc2)cc1OS(=O)(=O)C(F)(F)F]

3-Oxo-3-pyridin-3-yl-propionic acid ethyl ester: To nicotinic acid (20 g, 162.6 mmol) dissolved in dry THF was added CDI (30.95 g, 273.9 mmol) at 10° C. The mixture was stirred at RT for 1 h. In another flask the potassium salt of diethyl malonate (40.17 g, 245.1 mmol) and MgCl₂ (18.05 g, 189.59 mmol) were suspended in THF and heated to 50° C. for 4 h. The nicotinic acid/CDI mixture was then added to it and the entire mixture stirred at RT for 16 h. After completion, the mixture was quenched with water and extracted with EtOAc. The organic phase was washed with brine, dried over Na₂SO₄ and concentrated. The crude compound was purified by column chromatography using 30% EtOAc-Hexane as an eluent to give 3-oxo-3-pyridin-3-yl-propionic acid ethyl ester (7.8 g, 24.7%).

2-Ethyl-5-pyridin-3-yl-2H-pyrazol-3-ol: To 3-oxo-3-pyridin-3-yl-propionic acid ethyl ester (500 mg, 3.57 mmol) in AcOH was added ethylhydrazine oxalate (231.9 mg, 3.86 mmol) and the mixture refluxed for 16 h. After which, the AcOH was evaporated and crude mass neutralized with aq. Na₂CO₃ solution. Following extraction with EtOAc, the organic phase was washed with brine, dried over Na₂SO₄ and concentrated. The crude material was purified by column chromatography using 2% MeOH-DCM as an eluent to give 2-ethyl-5-pyridin-3-yl-2H-pyrazol-3-ol (110 mg, 22.5%) as a yellow solid.

Trifluoro-methanesulfonic acid 2-ethyl-5-pyridin-3-yl-2H-pyrazol-3-yl ester: To a solution of 2-ethyl-5-pyridin-3-yl-2,4-dihydro-pyrazol-3-one (200 mg, 1.058 mmol) in THF, cooled to 0° C., was added NaH (33 mg, 1.37 mmol) followed by N,N-bis(Trifluoromethanesulfonyl) aniline (567 mg, 1.58 mmol). The resulting mixture was stirred at 25° C. for 1 h, after which, it was quenched with ice-water and extracted with EtOAc. The organic phase was washed with 1 N NaOH, dried over Na₂SO₄ and concentrated. The crude material was then purified by column chromatography using 20% EtOAc-Hexane as an eluent to give trifluoro-methanesulfonic acid 2-ethyl-5-pyridin-3-yl-2H-pyrazol-3-yl ester (170 mg, 50%).

### Intermediate 3

### Trifluoro-methanesulfonic acid 2-ethyl-5-pyrazin-2-yl-2H-pyrazol-3-yl ester

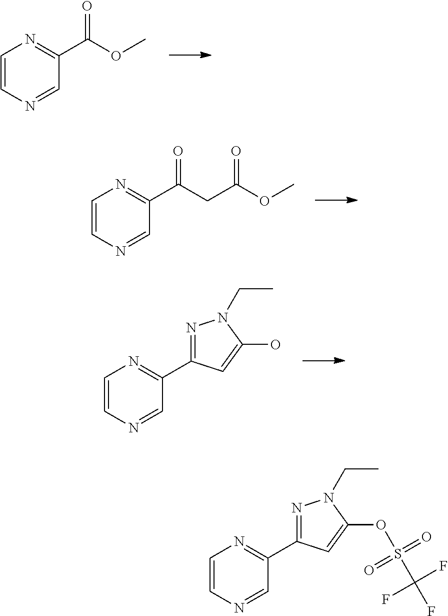

Methyl 3-oxo-3-(pyrazin-2-yl)propanoate: To a stirred solution of sodium methoxide (25% in MeOH, 27.54 mL, 72.4 mmol, 1 eq) in 90 mL of toluene at 110° C. in a 3-neck flask attached with a mechanical stirrer, condenser and dropping funnel was added a solution of methylpyrazine-2-carboxylate (10 g, 72.4 mmol, 1 eq) in 115 mL of methyl acetate, dropwise, over a period of ˜35-40 min. A yellow precipitate was formed. Stirring was continued at 110° C. for 3 hrs. The reaction was cooled and the yellow precipitate was filtered and washed with a small quantity of toluene. This solid was taken into 200 mL of saturated ammonium chloride and 400 mL of EtOAc. The aqueous layer was extracted twice with EtOAc. The combined organic layers were dried over magnesium sulfate, filtered and evaporated to give 6.52 g (50%) of methyl 3-oxo-3-(pyrazin-2-yl)propanoate as a yellow solid.

Ethyl-3-(pyrazin-2-yl)-1H-pyrazol-5-ol: Ethylhydrazine oxalate (6.89 g, 45.9 mmol, 1 eq) was stirred with 450 mL of anhydrous ethanol for 10 min. To this was added methyl 3-oxo-3-(pyrazin-2-yl)propanoate (8.27 g, 45.9 mmol, 1 eq) and the mixture was refluxed for 10 hrs. The reaction was cooled, evaporated, taken into 300 ml of EtOAc, extracted with water and brine, dried over anhydrous magnesium, filtered and evaporated to yield 8.7 g of 1-ethyl-3-(pyrazin-2-yl)-1H-pyrazol-5-ol as a red oil. This material was used without further purification.

Trifluoro-methanesulfonic acid 2-ethyl-5-pyrazin-2-yl-2H-pyrazol-3-yl ester: To a stirred solution of 1-ethyl-3-(pyrazin-2-yl)-1H-pyrazol-5-ol (8.7 g, 45.7 mmol, 1 eq) in 230 mL DMF at 0° C. was added NaH (2.93 g, 73.2 mmol, 1.6 eq). The mixture was allowed to warm to rt and stirred for 1 hr. 1,1,1-Trifluoro-N-phenyl-N-(trifluoromethylsulfonyl)methanesulfonamide (24.5 g, 68.6 mmol, 1.5 eq) was added and stirred at RT for 90 min. The mixture was cooled in an ice bath, quenched with saturated ammonium chloride, evaporated and taken into EtOAc, extracted with water and brine, dried over anhydrous magnesium sulfate, filtered and evaporated to an oil. Flash chromatography on silica gel (400 g) using a gradient of 10-30% EtOAC/hexane gave 9.27 g (62.9%) of trifluoro-methanesulfonic acid 2-ethyl-5-pyrazin-2-yl-2H-pyrazol-3-yl ester as a white solid. LC-MS (ES) calculated for C₁₀H₉F₃N₄O₃S, 322.27; found m/z 322.9 [M+H]⁺.

### Intermediate 4

### 3-(5-bromo-1-methyl-1H-[1,2,4]triazol-3-yl)-pyridine

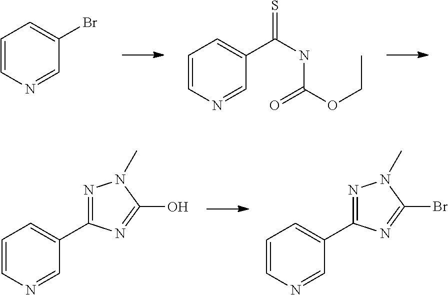

Ethyl pyridine-3-carbonothioylcarbamate: n-BuLi (2.5M in THF, 60 mL, 150 mmol, 1 eq) was charged into a 3-neck 2000 ml round bottom flask, attached with a mechanical stirrer and two dropping funnels (one containing a solution of 3-bromopyridine (14.46 mL, 150 mmol, 1 eq) in 220 ml of anhydrous ether and the other one containing O-ethyl carbonisothiocyanatidate (20.4 mL, 180 mmol, 1.2 eq) in 500 mL of anhydrous THF) under argon. The solution was cooled to −78° C. The 3-bromopyridine solution was added dropwise over 45 min and stirred at −7° C. for 30 min. The solution of O-ethyl carbonisothiocyanatidate was added dropwise over 75 min. Stirring was continued and the reaction mixture was allowed to come to RT overnight. 50 mL of saturated ammonium chloride was added and the reaction mixture was concentrated to small volume, diluted with EtOAc, washed with brine, dried over anhydrous magnesium sulfated, filtered and evaporated to a red oil. Flash chromatography on silica gel (600 g) using a gradient of 0-50% EtOAc/hexanes in 60 min gave 5.2 g (16.5%) of ethyl pyridine-3-carbonothioylcarbamate as a yellow solid. LC-MS (ES) calculated for C₉H₁₀N₂O₂S, 210.26; found m/z 211.1 [M+H]⁺.

methyl-3-(pyridin-3-yl)-1H-1,2,4-triazol-5-ol: The solution of ethyl pyridine-3-carbonothioylcarbamate (4.6 g, 21.9 mmol, 1 eq) and methylhydrazine (46 mL, 873 mmol, 39.9 eq) in 46 mL THF was heated at 80° C. in an oil bath for 40 min. The reaction mixture was cooled and evaporated. Flash chromatography on silica gel (240 g) using a gradient of 20-100% EtOAc/hexanes in 60 min gave 2.65 g (69%) of 1-methyl-3-(pyridin-3-yl)-1H-1,2,4-triazol-5-ol as an off-white solid. LC-MS (ES) calculated for C₈H₈N₄O, 176.18; found m/z 177.1 [M+H]⁺.

3-(5-bromo-1-methyl-1H-[1,2,4]triazol-3-yl)-pyridine: 1-methyl-3-(pyridin-3-yl)-1H-1,2,4-triazol-5-ol (1.2 g, 11.33 mmol, 1 eq) and phosphoryl tribromide (14.56 g, 50.84 mmol, 3.98 eq) were combined in a microwave reaction vessel and sealed. The mixture was heated at 120° C. in an oil bath for 2 hrs. The reaction mixture was cooled in acetone/dry ice bath and neutralized carefully with a saturated sodium bicarbonate solution, extracted with EtOAc, dried over anhydrous magnesium, filtered and evaporated. Flash chromatography on silica gel (120 g) using a gradient column of 0-60% EtOAc/hexane in 45 min gave 2.28 g (74%) of 3-(5-bromo-1-methyl-1H-[1,2,4]triazol-3-yl)-pyridine as a white solid. LC-MS (ES) calculated for C₈H₇BrN₄, 239.08; found m/z 240.0 [M+H]⁺.

### Intermediate 5

### 3-(5-Bromo-1-ethyl-1H-[1,2,4]triazol-3-yl)-pyridine

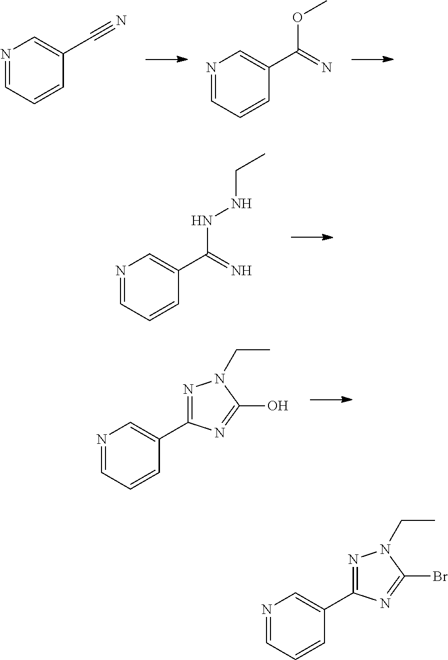

Nicotinimidic acid methyl ester: To a stirred solution of 3-cyanopyridine (5.0 g, 48.07 mmol) in methanol-1,4-dioxane (1:1; 50 ml) was added sodium methoxide (2.85 g, 52.88 mmol) at 0° C. The reaction mixture was stirred for 24 h at rt, after which the solvent was removed, and water (20 mL) was added to the resulting mass. This mixture was extracted with ethyl acetate (2×50), and the organic layers were dried, concentrated in vacuo and purified by column chromatography (20% EtOAc/Hexanes) to give nicotinimidic acid methyl ester (3.6 g, 55%) as light yellow liquid.

N′-ethylnicotinimidohydrazide: To a stirred solution of nicotinimidic acid methyl ester (2.0 g, 14.70 mmol) in dry pyridine (10 mL) was added ethyl hydrazine oxalate (2.34 g, 15.58 mmol) at rt. The mixture was stirred for 12 h, after which the solvent was removed to furnish a crude mass. This material was triturated with diethyl ether to give N′-ethylnicotinimidohydrazide (2.1 g, 87%) as a white solid.

2-Ethyl-5-pyridin-3-yl-2H-[1,2,4]triazol-3-ol: To a stirred solution of N′-ethylnicotinimidohydrazide (0.500 g, 3.05 mmol) in dry DMF (15 mL) was added CDI (0.524 g, 3.23 mmol) at rt. The mixture was then stirred for 12 h, after which the DMF was removed in vacuo, the material redissolved in methylene dichloride (25 mL), and filtered through a sintered funnel. The filtrate was concentrated under reduced pressure to provide a crude mass that was purified by column chromatography (20% methanol in DCM), to give 2-Ethyl-5-pyridin-3-yl-2H-[1,2,4]triazol-3-ol (0.200 g, 35%) as a white solid.

3-(5-Bromo-1-ethyl-1H-[1,2,4]triazol-3-yl)-pyridine: A solution of 2-Ethyl-5-pyridin-3-yl-2H-[1,2,4]triazol-3-ol (0.240 g, 1.26 mmol) in phosphorus oxybromide (1.44 g, 5.05 mmol) was stirred at 140° C. for 1 h. It was then cooled to 0° C. and the solution was basified to pH ˜9 with an aqueous solution of saturated sodium bicarbonate. The aqueous mixture was extracted with ethyl acetate (3×20 mL), and the organic layers were then dried over anhydrous sodium sulfate, concentrated, and purified by column chromatography (20% EtOAc/Hexanes) to give 3-(5-Bromo-1-ethyl-1H-[1,2,4]triazol-3-yl)-pyridine (0.160 g, 50.19%) as a brown solid.

### Intermediate 6

### Trifluoro-methanesulfonic acid 5-methyl-2-pyridin-2-yl-thiazol-4-yl ester

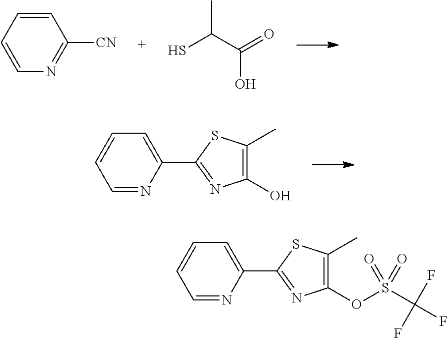

5-Methyl-2-pyridin-2-yl-thiazol-4-ol: To 2-cyanopyridine (5 g, 48 mmol) and thiolactic acid (5.1 g, 48 mmol) was added pyridine (0.97 mL, 12 mmol) and the mixture stirred at 100° C. After 3 h, the mixture was cooled to 25° C. and EtOH (50 mL) was added. After 30 min. the solvent was removed, and the residue washed with diethylether (3×30 mL) to give 5-Methyl-2-pyridin-2-yl-thiazol-4-ol (7 g, 76%).

Trifluoro-methanesulfonic acid 5-methyl-2-pyridin-2-yl-thiazol-4-yl ester: To a solution of 5-Methyl-2-pyridin-2-yl-thiazol-4-ol (500 mg, 2.6 mmol) in THF at 0° C. was added NaH (81.12 mg, 3.38 mmol) followed by N-phenyl bis(trifluoromethanesulfonimide) (1.08 g, 3.02 mmol). The reaction mixture was stirred at 25° C. for 1 h, after which water was added at 0° C. and the entire mixture extracted with EtOAc (3×20 mL). The organic phase was washed with brine, dried over Na₂SO₄, concentrated, and the crude compound was purified by column chromatography (10-20% EtOAc-Hexane) to give Trifluoro-methanesulfonic acid 5-methyl-2-pyridin-2-yl-thiazol-4-yl ester (200 mg, 24%).

### Intermediate 7

### Trifluoro-methanesulfonic acid 5-methyl-2-pyridin-3-yl-thiazol-4-yl ester

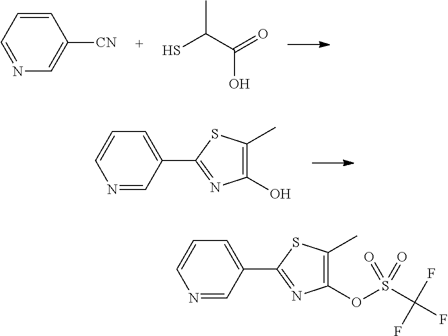

5-Methyl-2-pyridin-3-yl-thiazol-4-ol: To nicotinonitrile (2 g, 19.21 mmol) and 2-mercapto-propionic acid (2.04 g, 19.21 mmol) was added pyridine (0.38 ml, 4.80 mmol). The mixture heated to 100° C. After 3 h the mixture was cooled to rt, diluted with EtOH (20 ml) and stirred for 10 min. The resulting solid was filtered, washed with ether and dried under vacuum to give 5-methyl-2-pyridin-3-yl-thiazol-4-ol (2.5 g, 67.7%).

Trifluoro-methanesulfonic acid 5-methyl-2-pyridin-3-yl-thiazol-4-yl ester: To a solution of 5-methyl-2-pyridin-3-yl-thiazol-4-ol (300 mg, 1.56 mmol) in THF, cooled to 0° C., was added NaH (24 mg, 48.70 mmol) followed by N,N-bis(trifluoromethanesulfonyl)aniline (357 mg, 1.81 mmol). The mixture was stirred at 25° C. for 1 h, after which it was quenched with ice-water and extracted with EtOAc. The organic phase was washed with 1N NaOH, dried over Na₂SO₄ and concentrated. The crude compound was purified by column chromatography using 20% EtOAc-Hexane as an eluent to obtain trifluoro-methanesulfonic acid 5-methyl-2-pyridin-3-yl-thiazol-4-yl ester (200 mg, 40%).

### Intermediate 8

### Trifluoro-methanesulfonic acid 5-ethyl-2-pyridin-2-yl-thiazol-4-yl ester

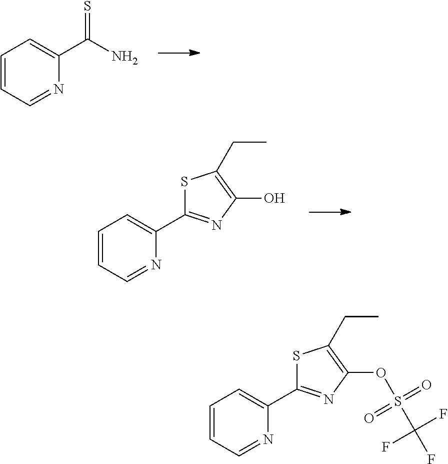 [SMILES: NC(=S)c1ccccn1 ; CCc1sc(-c2ccccn2)nc1O ; CCc1sc(-c2ccccn2)nc1OS(=O)(=O)C(F)(F)F]

5-Ethyl-2-pyridin-2-yl-thiazol-4-ol: To a stirred solution of pyridine-2-carbothioic acid amide (0.300 g, 2.17 mmol) and 2-bromo-butyric acid ethyl ester (0.33 mL, 2.61 mmol) in ethanol (7 mL) was added pyridine (0.3 mL, 3.69 mmol) at rt. The mixture was heated to reflux for 48 h (monitoring by TLC; ethyl acetate-hexane=1:1; Rf ˜0.3), after which it was concentrated under reduce pressure to provide a crude mass. This material was redissolved in MeOH, addition of diethyl ether caused precipitation of 5-ethyl-2-pyridin-2-yl-thiazol-4-ol (0.300 g, 67%) as brown solid. LC-MS: 207 [M+H].

Trifluoro-methanesulfonic acid 5-ethyl-2-pyridin-2-yl-thiazol-4-yl ester: To a stirred solution of 5-ethyl-2-pyridin-2-yl-thiazol-4-ol (750 mg, crude) in dry THF (50 mL) was added 60% NaH (145 mg, 3.64 mmol) at 0° C. The mixture was stirred for 15 min at this temperature before N,N-bis(trifluoromethylsulfonyl)aniline (1.3 g, 3.64 mmol) was added. The reaction mixture was allowed to warm to rt and stirring was continued for 1 h (monitoring by TLC; EtOAc-hexane=3:7; Rf ˜0.5), at which point it was quenched with aqueous NH₄Cl (˜5 mL), extracted with DCM (2×10 ml/mmol), dried, concentrated under reduced pressure, and purified by column chromatography (ethyl acetate-hexane=1:19 to 1:9) to give trifluoro-methanesulfonic acid 5-ethyl-2-pyridin-2-yl-thiazol-4-yl ester (300 mg; 41% two steps) as colorless oil.

### Intermediate 9

### Trifluoro-methanesulfonic acid 5-ethyl-2-pyridin-3-yl-thiazol-4-yl ester

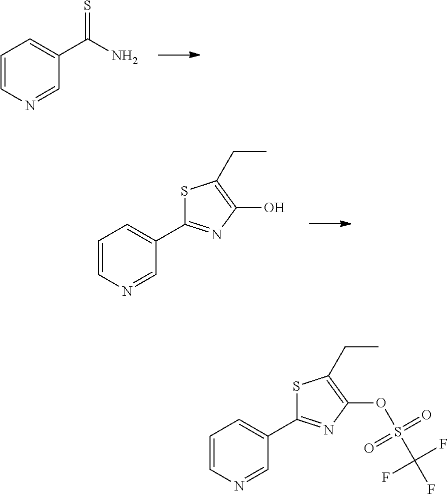 [SMILES: NC(=S)c1cccnc1 ; CCc1sc(-c2cccnc2)nc1O ; CCc1sc(-c2cccnc2)nc1OS(=O)(=O)C(F)(F)F]

Trifluoro-methanesulfonic acid 5-ethyl-2-pyridin-3-yl-thiazol-4-yl ester: To a solution of pyridine-3-carbothioamide (1 g, 7.24 mmol) in EtOH (15 mL) and pyridine (1 mL, 12.3 mmol) was added methyl 2-bromobutanoate (1 mL, 8.68 mmol). The mixture was heated at reflux for 18 hours, after which it was cooled and concentrated. The crude 5-Ethyl-2-pyridin-3-yl-thiazol-4-ol was then redissolved in DMF (36 mL) at 0° C., and to the mixture was added 60% sodium hydride (751 mg, 18.8 mmol). After stirring for 15 min at rt, N,N-bis(trifluoromethylsulfonyl)aniline (3.87 g, 10.8 mmol) was added. The mixture was reacted for 20 min, quenched with sat. NH₄Cl, diluted with diethyl ether. The mixture was washed with water, and then brine. The organic layer was concentrated, and the resulting material chromatographed (5-55% EtOAc/Hexanes to give trifluoro-methanesulfonic acid 5-ethyl-2-pyridin-3-yl-thiazol-4-yl ester (0.85 g) as an orange oil.

### Intermediate 10

### Trifluoro-methanesulfonic acid 5-methyl-2-pyrazin-2-yl-thiazol-4-yl ester

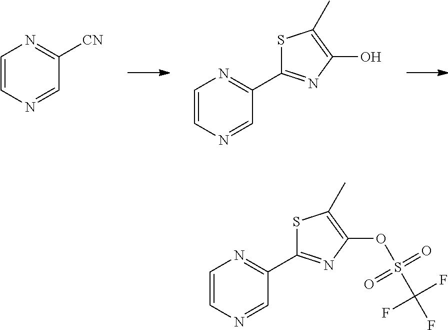 [SMILES: N#Cc1cnccn1 ; Cc1sc(-c2cnccn2)nc1O ; Cc1sc(-c2cnccn2)nc1OS(=O)(=O)C(F)(F)F]

5-Methyl-2-pyrazin-2-yl-thiazol-4-ol: In a 250 mL round-bottomed flask, pyrazine-2-carbonitrile (10 g, 95.1 mmol), pyridine (2.26 g, 2.33 ml, 28.5 mmol,) and 2-mercaptopropionic acid (10.1 g, 95.1 mmol) were combined to give a light yellow solution. The reaction mixture was heated to 100° C. and stirred for 2 h. Upon cooling, the thick yellow mixture was diluted with 100 mL ethanol and stirred for 30 min. The slurry was then filtered, and washed with diethyl ether (2×100 mL) to give 5-methyl-2-pyrazin-2-yl-thiazol-4-ol (17.86 g, 97.1%) as yellow solid which was used directly without further purification.

Trifluoro-methanesulfonic acid 5-methyl-2-pyrazin-2-yl-thiazol-4-yl ester: In a 500 mL round-bottomed flask, 5-methyl-2-(pyrazin-2-yl)thiazol-4-ol (12.24 g, 63.3 mmol) was cooled to 0° C. in THF (110 ml) and stirred for 33 min. 60% sodium hydride (3.32 g, 83.0 mmol) was added followed by N-phenylbis(trifluoromethanesulfonimide) (26.6 g, 72.8 mmol) and the resultant reaction mixture was warmed to 25° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 120 g, 25% to 45% ethyl acetate in hexanes) to give trifluoro-methanesulfonic acid 5-methyl-2-pyrazin-2-yl-thiazol-4-yl ester (7.45 g, 36.2%) as a colorless oil which solidified to an off-white solid.

### Intermediate 11

### Trifluoro-methanesulfonic acid 5-ethyl-2-pyrazin-2-yl-thiazol-4-yl ester

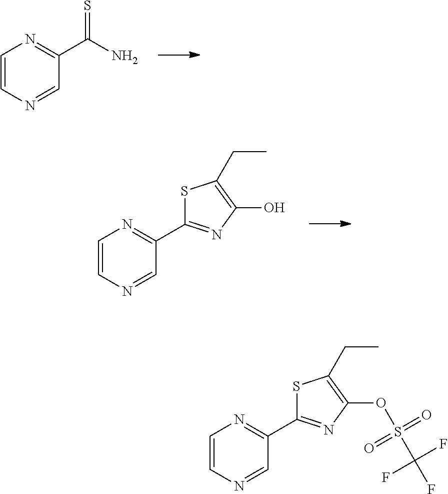 [SMILES: NC(=S)c1cnccn1 ; CCc1sc(-c2cnccn2)nc1O ; CCc1sc(-c2cnccn2)nc1OS(=O)(=O)C(F)(F)F]

5-Ethyl-2-pyrazin-2-yl-thiazol-4-ol: A solution of pyrazine-2-carbothioamide (1 g, 7.19 mmol) in ethanol (20 ml) was treated with methyl 2-bromobutyrate (1.56 g, 992 μl, 8.62 mmol) an pyridine (853 mg, 872 μl, 10.8 mmol) and heated to reflux for 2 hours. The reaction mixture was cooled and concentrated to dryness under reduced pressure, and the resulting solid was filtered and washed with diethyl ether to provide 5-ethyl-2-pyrazin-2-yl-thiazol-4-ol (0.740 g, 50%) which was used directly without further purification. MS (M+H)=208.

Trifluoro-methanesulfonic acid 5-ethyl-2-pyrazin-2-yl-thiazol-4-yl ester: In a 100 mL round-bottomed flask, 5-ethyl-2-(pyrazin-2-yl)thiazol-4-ol (0.74 g, 3.57 mmol) was cooled to 0° C. in THF (110 ml) and stirred for 30 min. 60% sodium hydride (0.187 g, 4.68 mmol) was added followed by N-phenylbis(trifluoromethanesulfonimide) (1.5 g, 4.11 mmol) and the resultant reaction mixture was warmed to 25° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 120 g, 20% to 25% ethyl acetate in hexanes) to give trifluoro-methanesulfonic acid 5-ethyl-2-pyrazin-2-yl-thiazol-4-yl ester (0.34 g, 28.1%) as light yellow oil which solidified upon standing.

### Intermediate 12

### Trifluoro-methanesulfonic acid 5-ethyl-2-pyridin-4-yl-thiazol-4-yl ester

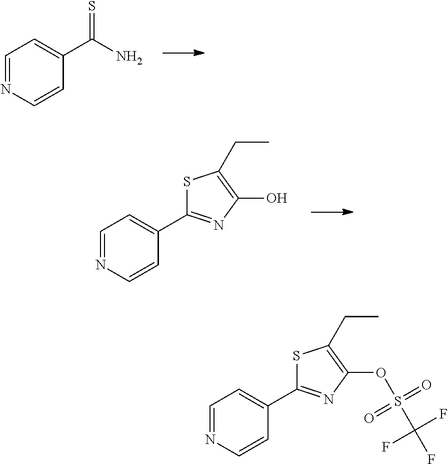 [SMILES: NC(=S)c1ccncc1 ; CCc1sc(-c2ccncc2)nc1O ; CCc1sc(-c2ccncc2)nc1OS(=O)(=O)C(F)(F)F]

5-Ethyl-2-pyridin-4-yl-thiazol-4-ol: To a stirred solution of thioisonicotinamide (0.500 g, 3.62 mmol) and 2-bromo-butyric acid ethyl ester (0.55 mL, 4.34 mmol) in ethanol (15 mL) was added pyridine (0.5 mL, 6.15 mmol) at rt. The mixture was refluxed for 72 h (monitoring by TLC; ethyl acetate-hexane=1:1; Rf ˜0.6), before being concentrated under reduce pressure. The crude material was then redissolved in MeOH and diethyl ether which caused the precipitation of pure 5-ethyl-2-pyridin-4-yl-thiazol-4-ol (0.350 g, 47%) that was isolated as a yellow solid. LC-MS: 207 [M+H].

Trifluoro-methanesulfonic acid 5-ethyl-2-pyridin-4-yl-thiazol-4-yl ester: To a stirred solution of 5-ethyl-2-pyridin-4-yl-thiazol-4-ol (0.350 g, 1.7 mmol) in dry THF (100 mL) was added NaH (0.040 g, 1.7 mmol) at 0° C. The reaction mixture was stuirred at this temperature for 15 min before N,N-bis(trifluoromethylsulfonyl)aniline (0.909 g, 2.55 mmol) was added. The mixture was then allowed to warm to rt and stirring was continued for 1 h (monitoring by TLC; EtOAc-hexane=3:7; Rf ˜0.6), at which point it was quenched with aqueous NH₄Cl (˜5 mL). The mixture was extracted with DCM (3×10 ml), dried, concentrated under reduced reaction, and purified by column chromatography (ethyl acetate-hexane=1:19 to 1:9) to give trifluoro-methanesulfonic acid 5-ethyl-2-pyridin-2-yl-thiazol-4-yl ester (0.200 g, 35%) as yellow oil. LC-MS: 339 [M+H].

### Intermediate 13

### Trifluoro-methanesulfonic acid 5-ethyl-2-(2-ethyl-pyridin-4-yl)-thiazol-4-yl ester

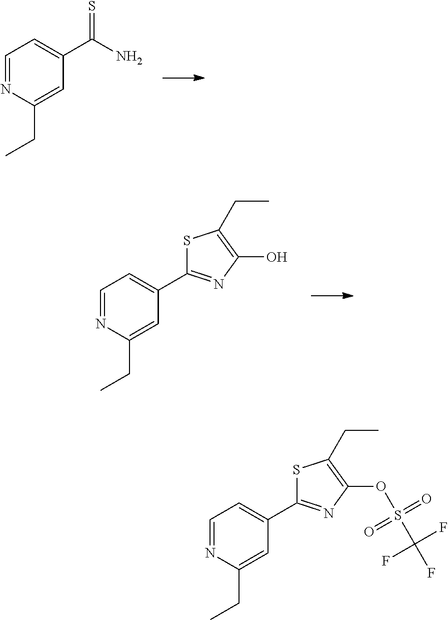 [SMILES: CCc1cc(C(N)=S)ccn1 ; CCc1cc(-c2nc(O)c(CC)s2)ccn1 ; CCc1cc(-c2nc(OS(=O)(=O)C(F)(F)F)c(CC)s2)ccn1]

5-Ethyl-2-(2-ethyl-pyridin-4-yl)-thiazol-4-ol: To a stirred solution of 2-ethyl-thioisonicotinamide (0.500 g, 3.01 mmol) and 2-bromo-butyric acid ethyl ester (0.46 mL, 3.61 mmol) in ethanol (15 mL) was added pyridine (0.4 mL, 6.15 mmol) at rt. The mixture was refluxed for 16 h (monitoring by TLC; ethyl acetate-hexane=1:1; Rf ˜0.6) before being concentrated under reduce pressure. The crude material was then redissolved in MeOH and diethyl ether which caused the precipitation of pure 5-Ethyl-2-ethyl-pyridin-4-yl-thiazol-4-ol (0.120 g, 17%) that was isolated as a yellow solid. LC-MS: 235 [M+H].

Trifluoro-methanesulfonic acid 5-ethyl-2-(2-ethyl-pyridin-4-yl)-thiazol-4-yl ester: To a stirred solution of 5-ethyl-2-(2-ethyl-pyridin-4-yl)-thiazol-4-ol (0.150 g, 0.64 mmol) in dry THF (50 mL) was added NaH (0.026 g, 0.64 mmol) at 0° C. The reaction mixture was stirred at this temperature for 15 min before N,N-bis(trifluoromethylsulfonyl)aniline (0.343.27 g, 0.96 mmol) was added. The mixture was then allowed to warm to rt and stirring was continued for 1 h (monitoring by TLC; EtOAc-hexane=3:7; Rf ˜0.3), at which point it was quenched with aqueous NH₄Cl (˜5 mL). The mixture was extracted with DCM (3×10 ml), dried, concentrated under reduced reaction, and purified by column chromatography (ethyl acetate-hexane=1:19 to 1:9) to give trifluoro-methanesulfonic acid 5-ethyl-2-(2-ethyl-pyridin-4-yl)-thiazol-4-yl ester (0.180 g, 77%) as viscous oil. LC-MS: 367 [M+H].

### Intermediate 14

### Trifluoro-methanesulfonic acid 5-ethyl-2-oxazol-2-yl-thiazol-4-yl ester

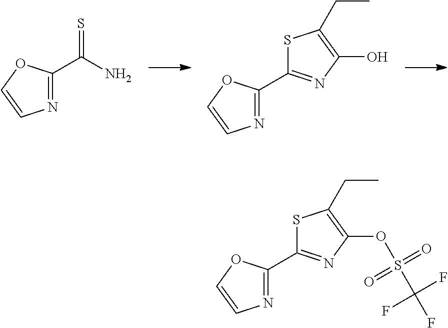 [SMILES: NC(=S)c1ncco1 ; CCc1sc(-c2ncco2)nc1O ; CCc1sc(-c2ncco2)nc1OS(=O)(=O)C(F)(F)F]

5-Ethyl-2-oxazol-2-yl-thiazol-4-ol: To a stirred solution of oxazole-2-carbothioic acid amide (0.300 g, 2.34 mmol) and 2-bromo-butyric acid ethyl ester (0.42 mL, 2.81 mmol) in ethanol (15 mL) was added pyridine (0.32 mL, 3.98 mmol) at rt. The mixture was refluxed for 16 h (monitoring by TLC; ethyl acetate-hexane=1:1; Rf ˜0.5) before being concentrated under reduce pressure. The crude material was then redissolved in MeOH and diethyl ether which caused the precipitation of pure 5-ethyl-2-oxazol-2-yl-thiazol-4-ol (0.200 g, 44%) that was isolated as a yellow solid. LC-MS: 197 [M+H].

Trifluoro-methanesulfonic acid 5-ethyl-2-oxazol-2-yl-thiazol-4-yl Ester: To a stirred solution of 5-ethyl-2-(5-methyl-isoxazol-3-yl)-thiazol-4-ol (0.250 g, 1.28 mmol) in dry THF (100 mL) was added NaH (0.051 g, 1.28 mmol) at 0° C. The reaction mixture was stuirred at this temperature for 15 min before N,N-bis(trifluoromethylsulfonyl)aniline (0.683 g, 1.91 mmol) was added. The mixture was then allowed to warm to rt and stirring was continued for 1 h (monitoring by TLC; EtOAc-hexane=3:7; Rf ˜0.3), at which point it was quenched with aqueous NH₄Cl (˜5 mL). The mixture was extracted with DCM (3×10 ml), dried, concentrated under reduced reaction, and purified by column chromatography (ethyl acetate-hexane=1:19 to 1:9) to give trifluoro-methanesulfonic acid 5-ethyl-2-oxazol-2-yl-thiazol-4-yl ester (0.200 g, 48%) as a viscous oil. LC-MS: 329 [M+H].

### Intermediate 15

### Trifluoro-methanesulfonic acid 5-ethyl-2-(5-methyl-isoxazol-3-yl)-thiazol-4-yl ester

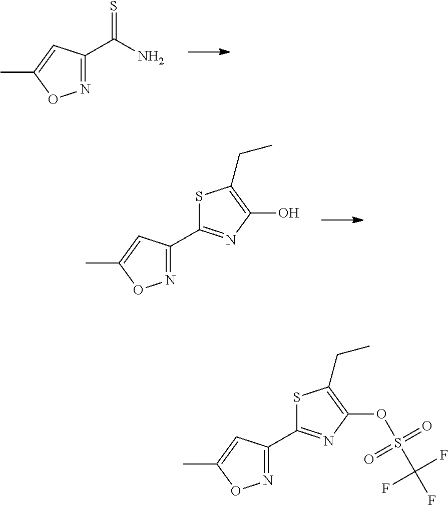

5-Ethyl-2-(5-methyl-isoxazol-3-yl)-thiazol-4-ol: To a stirred solution of 5-methyl-isoxazole-3-carbothioic acid amide (0.300 g, 2.11 mmol) and 2-bromo-butyric acid ethyl ester (0.0.37 mL, 2.53 mmol) in ethanol (10 mL) was added pyridine (0.29 mL, 3.59 mmol) at rt. The mixture was refluxed for 16 h (monitoring by TLC; ethyl acetate-hexane=3:7; Rf ˜0.3) before being concentrated under reduce pressure. The crude material was then redissolved in MeOH and diethyl ether which caused the precipitation of pure 5-ethyl-2-(5-methyl-isoxazol-3-yl)-thiazol-4-ol (0.430 g, crude) that was isolated as a brown sticky solid. LC-MS: 197 [M+H].

Trifluoro-methanesulfonic acid 5-ethyl-2-(5-methyl-isoxazol-3-yl)-thiazol-4-yl ester: To a stirred solution of 5-ethyl-2-(5-methyl-isoxazol-3-yl)-thiazol-4-ol (0.430 g, 2.05 mmol) in dry THF (50 mL) was added 60% NaH (85 mg, 2.05 mmol) at 0° C.

The reaction mixture was stirred at this temperature for 15 min before N,N-bis(trifluoromethylsulfonyl)aniline (0.731 g, 2.05 mmol) was added. The mixture was then allowed to warm to rt and stirring was continued for 2 h (monitoring by TLC; EtOAc-hexane=3:7; Rf ˜0.4), at which point it was quenched with aqueous NH₄Cl (˜5 mL). The mixture was extracted with DCM (3×10 ml), dried, concentrated under reduced reaction, and purified by column chromatography (ethyl acetate-hexane=1:19 to 1:9) to give trifluoro-methanesulfonic acid 5-ethyl-2-(5-methyl-isoxazol-3-yl)-thiazol-4-yl ester (0.140 g, 33%) as viscous oil. LC-MS: 329 [M+H].

### Intermediate 16

### 5-Bromo-2-ethanesulfonyl-4-methyl-pyridine

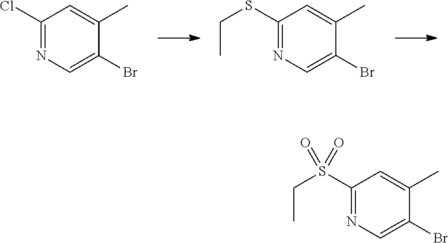

5-bromo-2-(ethylthio)-4-methylpyridine: To a mixture of 5-bromo-2-chloro-4-picoline (1 g, 4.84 mmol, Eq: 1.00) and sodium ethanethiolate (530 mg, 6.3 mmol, Eq: 1.3) was added NMP (10 ml). The mixture was placed in a microwave vial and irradiated at 150° C. for 30 min. Upon completion, the reaction mixture was then poured into water and extracted with EtOAc. The organic layer was washed with brine, dried over Na₂SO₄, concentrated under reduced pressure, and the crude material purified by column chromatography (EtOAc/Hex, 0 to 5%) to give 5-bromo-2-(ethylthio)-4-methylpyridine (1 g, 89%) as a colorless oil.

5-bromo-2-ethanesulfonyl-4-methyl-pyridine: To a solution of 5-bromo-2-(ethylthio)-4-methylpyridine (1 g, 4.31 mmol, Eq: 1.00) in THF (10 ml), was added Oxone (3.97 g, 6.46 mmol, Eq: 1.5) in water (10 ml). The reaction mixture was stirred at room temperature for 1.5 days, then poured into an additional amount of water, and the mixture extracted with EtOAc. The organic layer was washed with brine, dried over Na₂SO₄, concentrated under reduced pressure, and the crude material purified by column chromatography (EtOAc/Hex, 10 to 40%) to give 5-bromo-2-ethanesulfonyl-4-methyl-pyridine (930 mg, 82%) as a white solid.

### Intermediate 17

### 5-(5-bromo-4-methylpyridin-2-yl)pyrimidin-2-amine

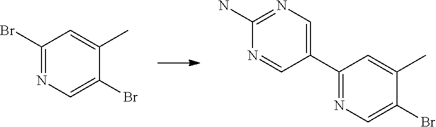

5-(5-bromo-4-methylpyridin-2-yl)pyrimidin-2-amine: To a mixture of 2,5-dibromo-4-methylpyridine (550 mg, 2.19 mmol, Eq: 1.00), 2-aminopyrimidin-5-ylboronic acid (365 mg, 2.63 mmol, Eq: 1.2), tetrakis(triphenylphosphine)palladium (0) (253 mg, 219 μmol, Eq: 0.1) and potassium carbonate (909 mg, 6.58 mmol, Eq: 3) was added dioxane (39.0 ml) and water (9.74 ml). The reaction mixture was then heated to 95° C. for 2 hrs. Upon completion, the mixture was concentrated under reduced pressure, and the crude material purified by chromatography using a (MeOH/DCM gradient) to give 5-(5-bromo-4-methylpyridin-2-yl)pyrimidin-2-amine (478 mg, 1.8 mmol, 82% yield) as an off-white solid.

### Intermediate 18

### 5-(5-bromo-4-methylpyridin-2-yl)-2-methylthiazole

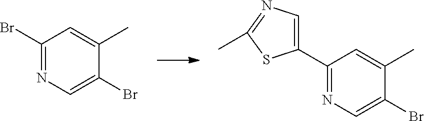

5-(5-bromo-4-methylpyridin-2-yl)-2-methylthiazole: To a mixture of 2,5-dibromo-4-methylpyridine (110 mg, 438 μmol, Eq: 1.00), 2-methyl-5-(4,4,5,5-tetramethyl-1,3,2-dioxaborolan-2-yl)thiazole (98.7 mg, 438 μmol, Eq: 1.00), tetrakis(triphenylphosphine)palladium (0) (50.7 mg, 43.8 μmol, Eq: 0.1) and potassium carbonate (182 mg, 1.32 mmol, Eq: 3) was added dioxane (7.79 ml) and water (1.95 ml). The reaction mixture was then heated to 95° C. for 3 hrs. Upon completion, the mixture was concentrated under reduced pressure, and the crude material purified by chromatography using (EtOAc/Hex gradient) to give 5-(5-bromo-4-methylpyridin-2-yl)-2-methylthiazole (76 mg, 282 mmol, 64% yield) as an off-white solid.

### Intermediate 19

### 5-bromo-4-methyl-2-(2-methyl-2H-tetrazol-5-yl)pyridine

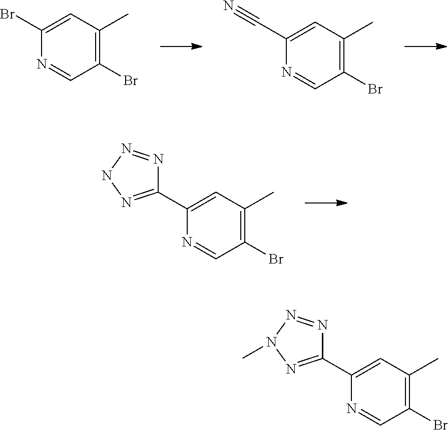

5-bromo-4-methyl-pyridine-2-carbonitrile: To a solution of 2,5-dibromo-4-methylpyridine (15 g, 59.8 mmol, Eq: 1.00) in DMF (100 ml) was added copper(I) cyanide (4.28 g, 47.8 mmol, Eq: 0.8) and sodium cyanide (2.34 g, 47.8 mmol, Eq: 0.8). The reaction mixture was refluxed for 20 hr at which point a precipitate formed. Upon cooling water was added and the mixture sonicated. The solids were filtered and washed with water. The resulting filtrate was extracted with EtOAc and the organic layers then combined, washed with water and brine, and concentrated under reduced pressure. The crude material was then purified by column chromatography (0-10% EtOAc/Hex gradient) to give 5-bromo-4-methyl-pyridine-2-carbonitrile (5 g, 42.4% yield) as a white solid.

5-bromo-4-methyl-2-(2H-tetrazol-5-yl)-pyridine: To a mixture of 5-bromo-4-methylpicolinonitrile (2.5 g, 12.7 mmol, Eq: 1.00), sodium azide (1.07 g, 16.5 mmol, Eq: 1.3) and triethylamine hydrochloride (2.27 g, 16.5 mmol, Eq: 1.3) was added xylene (25 ml). The reaction mixture was heated at 140° C. overnight, and upon cooling was concentrated under reduced pressure. To the white residue was added water and an aq. HCl solution. These solids were filtered, washed with water, and dried to give 5-bromo-4-methyl-2-(2H-tetrazol-5-yl)-pyridine (2.43 g, 80% yield) as an off-white solid.

5-bromo-4-methyl-2-(2-methyl-2H-tetrazol-5-yl)pyridine: To a solution of 5-bromo-4-methyl-2-(2H-tetrazol-5-yl)-pyridine (900 mg, 3.75 mmol, Eq: 1.00) in THF (18.0 ml) was added trimethylsilyldiazomethane (4.12 ml, 8.25 mmol, Eq: 2.2) dropwise at room temperature. The reaction mixture was stirred for 3 hours, then diluted with water and EtOAc. The organic layer was separated washed with H₂O and brine, concentrated under reduced pressure, and purified by chromatography (0-5% MeOH/DCM) to give 5-bromo-4-methyl-2-(2-methyl-2H-tetrazol-5-yl)pyridine (530 mg, 2.09 mmol, 55.6% yield) as a white solid.

### Intermediate 20

### 2-(3-Bromo-4-methyl-phenyl)-oxazole

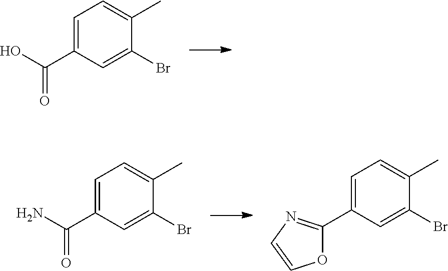

3-Bromo-4-methyl-benzamide: To a stirred solution of 3-bromo-4-methyl-benzoic acid (2.0 g, 9.48 mmol) in DCM-DMF (4:1; 25 mL) was added oxalyl chloride (0.89 mL, 9.48 mmol) at 0° C. Stirring was continued for another 3 h (monitoring by TLC; EtOAc-hexane=3:7 as eluent, Rf=0.3), at which point the volatiles were concentrated under reduced pressure. The residue was redissolved in DCM (20 mL) and ammonium hydroxide (10 mL) was added to the mixture at 0° C. Stirring was continued at 0° C. for another 4 h (monitoring by TLC, ethyl acetate-hexane=3:7, Rf=0.3), at which point the solid formed was filtered to give 3-bromo-4-methyl-benzamide (2.0 g, 98.6%) as an off-white solid.

2-(3-Bromo-4-methyl-phenyl)-oxazole: To 3-bromo-4-methyl-benzamide (1.0 g, 4.67 mmol) and vinylene carbonate (0.4 ml, 0.6.30 mmol) was added PPA (−15 mL) at rt. The mixture was then heated to 170° C. for 3 h (monitoring by TLC, EtOAc-hexane=3:7, Rf=0.4), before being quenched with water (40 mL) and extracted with EtOAc (2×20 ml). The combined extracts were washed with brine (40 mL), dried, concentrated under reduced pressure, and purified by column chromatography (EtOAc-hexane=1:19 to 1:7) to give 2-(3-Bromo-4-methyl-phenyl)-oxazole (0.4 g, 36.0%) as a white solid.

### Intermediate 21

### Trifluoro-methanesulfonic acid 5-methyl-2-pyridin-4-yl-thiazol-4-yl ester

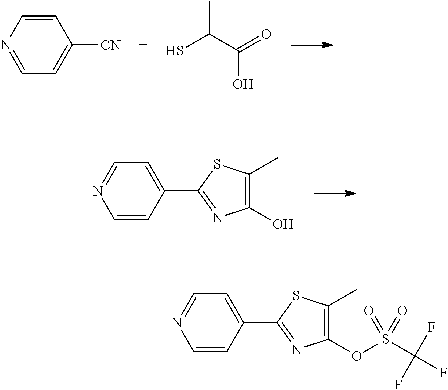

5-Methyl-2-pyridin-4-yl-thaizol-4-ol: To 4-cyanopyridine (5 g, 48 mmol) and thiolactic acid (5.1 g, 48 mmol) was added pyridine (0.97 mL, 12 mmol) and the mixture stirred at 100° C. Upon completion, the mixture was cooled to 25° C. and EtOH (50 mL) was added and stirred for 30 min. The resulting solids were filtered and washed with Et₂O (3×30 mL) to give 5-Methyl-2-pyridin-4-yl-thiazol-4-ol (7 g, 76%).

Trifluoro-methanesulfonic acid-5-methyl-2-pyridin-4-yl-thiazol-4-yl ester: To a solution of 5-Methyl-2-pyridin-4-yl-thiazol-4-ol (4 g, 20.8 mmol) in THF at 0° C. and added NaH (0.65 g, 24.14 mmol) followed by N-phenyl bis(trifluoromethanesulfonimide) (8.62 g, 27.1 mmol). The mixture was stirred at 25° C. for 1 h, after which water was added at 0° C. The mixture was extracted with EtOAc (3×20 mL) and then the organic phase was washed with brine, dried over Na₂SO₄, and concentrated. The crude compound was purified by column chromatography (10-20% EtOAc-Hexane) to give Trifluoro-methanesulfonic acid 5-methyl-2-pyridin-4-yl-thiazol-4-yl ester (4.5 g, 67%).

### Part II. Preparation of Certain Embodiments of the Invention

### Example 1

### 3-(2,6-Difluoro-phenyl)-7-(2-methyl-4-trifluoromethyl-phenyl)-3,4-dihydro-2H-benzo[1,4]oxazine

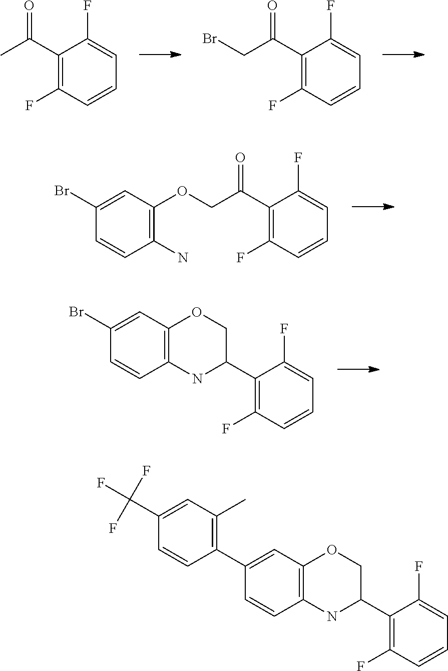 [SMILES: CC(=O)c1c(F)cccc1F ; O=C(CBr)c1c(F)cccc1F ; Nc1ccc(Br)cc1OCC(=O)c1c(F)cccc1F ; Fc1cccc(F)c1C1COc2cc(Br)ccc2N1 ; Cc1cc(C(F)(F)F)ccc1-c1ccc2c(c1)OCC(c1c(F)cccc1F)N2]

2-Bromo-1-(2,6-difluoro-phenyl)-ethanone: To a stirred solution of 1-(2,6-difluoro-phenyl)-ethanone (1.0 g, 6.4 mmol) in diethyl ether (14 mL), was added anhydrous aluminium chloride (0.009 g, 0.064 mmol) at rt. After 5 min the reaction mixture was cooled to 0° C., and bromine (0.33 mL, 6.4 mmol) was added dropwise. Stirring was continued for 2 h at rt (monitoring by TLC; ethyl acetate/hexane=1:9; Rf ˜0.6), at which point the reaction mixture was poured onto crushed ice and extracted with EtOAc (3×20 mL). The organic layer was washed with aqueous sodium thiosulphate (20 mL) and brine (10 mL), dried, and concentrated under reduce pressure to give 2-bromo-1-(2,6-difluoro-phenyl)-ethanone (1.3 g, 95%) as yellow oil which was sufficiently pure for the next step.

2-(2-Amino-5-bromophenoxy)-1-(2,6-difluoro-phenyl)-ethanone: To a stirred suspension of 2-bromo-1-(2,6-difluoro-phenyl)-ethanone (1.0 g, 4.26 mmol) and Cs₂CO₃ (1.4 g, 4.26 mmol) in acetonitrile (40 mL) was added 2-amino-5-bromophenol (0.800 g, 4.26 mmol) at rt. The reaction mixture was stirred for 3 h (monitoring by TLC; ethyl acetate-hexane=1:9; Rf ˜0.5) before water (60 mL) was added. The aqueous layer was then extracted with EtOAc (3×30 mL), dried, concentrated under reduce pressure, and purified by column chromatography (EtOAc-hexane=1:49 to 1:19) to give 2-(2-amino-5-bromophenoxy)-1-(2,6-difluoro-phenyl)-ethanone (1.0 g, 69%) as an off-white solid.

7-Bromo-3-(2,6-difluoro-phenyl)-3,4-dihydro-2H-benzo[1,4]oxazine: To a stirred solution of 2-(2-amino-5-bromophenoxy)-1-(2,6-difluoro-phenyl)-ethanone (0.600 g, 1.7 mmol) and NaBH(OAc)₃ (0.744 g, 3.51 mmol) in dry DCM (25 mL) was added TFA (0.13 mL, 1.75 mmol) at rt. The mixture was stirred for 3 h (monitoring by TLC; ethyl acetate-hexane=1:9; Rf ˜0.4), before being quenched with a saturated solution of NaHCO₃. The aqueous layer was then extracted with DCM (2×15 mL), dried, concentrated under reduce pressure, and purified by column chromatography (ethyl acetate-hexane=1:49 to 1:19) to give 7-bromo-3-(2,6-difluoro-phenyl)-3,4-dihydro-2H-benzo[1,4]oxazine (550 mg, 96%). LC-MS: 327 [M+H].

3-(2,6-Difluoro-phenyl)-7-(2-methyl-4-trifluoromethyl-phenyl)-3,4-dihydro-2H-benzo[1,4]oxazine: To a stirred solution of 7-bromo-3-(2,6-difluoro-phenyl)-3,4-dihydro-2H-benzo[1,4]oxazine (0.1 g, 0.31 mmol) in 1,4-dioxane (10 mL) was added 1M K₂CO₃ (0.9 mL), Pd(PPh₃)₄(0.035 g, 0.3 mmol) and 2-methyl-4-trifluoromethyl phenylboronic acid (0.63 g, 0.31 mmol) at rt. The mixture was then heated to 85° C. for 16 h under nitrogen (monitoring by TLC; ethyl acetate-hexane=1:9; Rf ˜0.5), cooled, and filtered through celite. The filtrate was concentrated under reduced pressure and purified by HPLC to give pure 3-(2,6-difluoro-phenyl)-7-(2-methyl-4-trifluoromethyl-phenyl)-3,4-dihydro-2H-benzo[1,4]oxazine (0.04 g, 32%) as an off-white solid. LC-MS: 406 [M+H].

### Example 2

### (R)-3-(2,6-Difluoro-phenyl)-7-(2-methyl-4-trifluoromethyl-phenyl)-3,4-dihydro-2H-benzo[1,4]oxazine or (S)-3-(2,6-Difluoro-phenyl)-7-(2-methyl-4-trifluoromethyl-phenyl)-3,4-dihydro-2H-benzo[1,4]oxazine

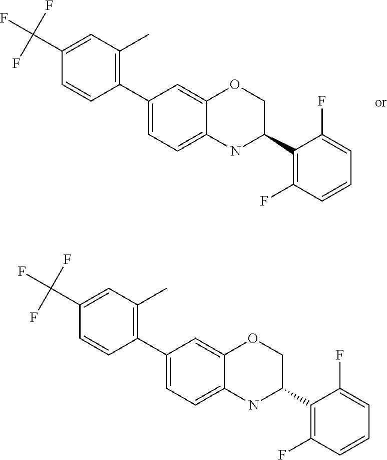 [SMILES: Cc1cc(C(F)(F)F)ccc1-c1ccc2c(c1)OC[C@@H](c1c(F)cccc1F)N2 ; Cc1cc(C(F)(F)F)ccc1-c1ccc2c(c1)OC[C@H](c1c(F)cccc1F)N2]

(R)-3-(2,6-Difluoro-phenyl)-7-(2-methyl-4-trifluoromethyl-phenyl)-3,4-dihydro-2H-benzo[1,4]oxazine or (S)-3-(2,6-Difluoro-phenyl)-7-(2-methyl-4-trifluoromethyl-phenyl)-3,4-dihydro-2H-benzo[1,4]oxazine: Was obtained as a pure enantiomer of unknown stereochemical configuration from chiral HPLC separation of Example 1.

### Example 3

### (S)-3-(2,6-Difluoro-phenyl)-7-(2-methyl-4-trifluoromethyl-phenyl)-3,4-dihydro-2H-benzo[1,4]oxazine or (R)-3-(2,6-Difluoro-phenyl)-7-(2-methyl-4-trifluoromethyl-phenyl)-3,4-dihydro-2H-benzo[1,4]oxazine

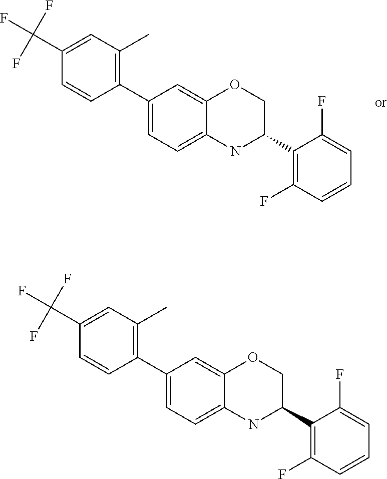 [SMILES: Cc1cc(C(F)(F)F)ccc1-c1ccc2c(c1)OC[C@H](c1c(F)cccc1F)N2 ; Cc1cc(C(F)(F)F)ccc1-c1ccc2c(c1)OC[C@@H](c1c(F)cccc1F)N2]

(S)-3-(2,6-Difluoro-phenyl)-7-(2-methyl-4-trifluoromethyl-phenyl)-3,4-dihydro-2H-benzo[1,4]oxazine or (R)-3-(2,6-Difluoro-phenyl)-7-(2-methyl-4-trifluoromethyl-phenyl)-3,4-dihydro-2H-benzo[1,4]oxazine: Was obtained as a pure enantiomer of unknown stereochemical configuration from chiral HPLC separation of Example 1.

### Example 4

### 3-(2,6-difluoro-phenyl)-7-(2-methyl-5-trifluoromethyl-phenyl)-3,4-dihydro-2H-benzo[1,4]oxazine

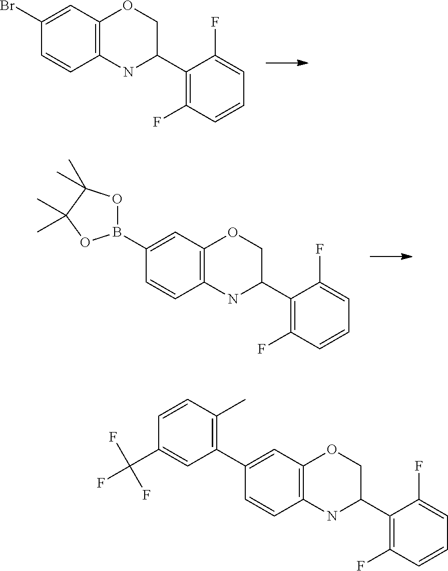 [SMILES: Fc1cccc(F)c1C1COc2cc(Br)ccc2N1 ; CC1(C)OB(c2ccc3c(c2)OCC(c2c(F)cccc2F)N3)OC1(C)C ; Cc1ccc(C(F)(F)F)cc1-c1ccc2c(c1)OCC(c1c(F)cccc1F)N2]

3-(2,6-difluoro-phenyl)-7-(4,4,5,5-tetramethyl-[1,3,2]dioxaborolan-2-yl)-3,4-dihydro-2H-benzo[1,4]oxazine: To a stirred solution of 7-bromo-3-(2,6-difluoro-phenyl)-3,4-dihydro-2H-benzo[1,4]oxazine (0.700 g, 2.15 mmol.) in 1,4-dioxane (25 mL) was added Pd(dppf)Cl₂.DCM (0.175 g, 0.21 mmol), KOAc (0.631 g, 6.44 mmol), and bis-pinacolato diborane (1.09 g, 4.29 mmol) at rt. The mixture was then heated to 85° C. for 16 h (monitoring by TLC; ethyl acetate-hexane=1:9; Rf ˜0.3), cooled, and filtered through celite. The filtrate was concentrated under reduced pressure and purified on alumina (ethyl acetate-hexane=1:49 to 1:19) to give 3-(2,6-difluoro-phenyl)-7-(4,4,5,5-tetramethyl-[1,3,2]dioxaborolan-2-yl)-3,4-dihydro-2H-benzo[1,4]oxazine (0.570 g, 71.13%) as an off-white solid. LC-MS: 374 [M+H].

3-(2,6-Difluoro-phenyl)-7-(2-methyl-5-trifluoromethyl-phenyl)-3,4-dihydro-2H-benzo[1,4]oxazine: To a stirred solution of 3-(2,6-difluoro-phenyl)-7-(4,4,5,5-tetramethyl-[1,3,2]dioxaborolan-2-yl)-3,4-dihydro-2H-benzo[1,4]oxazine (0.1 g, 0.27 mmol) in 1,4-dioxane (5 mL) was added 1M K₂CO₃ (0.8 mL), Pd(PPh₃)₄, (0.031 g, 0.03 mmol), SPhos (0.006 g, 0.01 mmol) and 2-bromo-1-methyl-4-trifluoromethyl-benzene (0.065 g, 0.27 mmol) at rt. The mixture was then heated to 85° C. for 16 h (monitoring by TLC; ethyl acetate-hexane=1:9; Rf ˜0.3), cooled, and filtered through celite. The filtrate was concentrated under reduced pressure and purified by HPLC to give 3-(2,6-difluoro-phenyl)-7-(2-methyl-5-trifluoromethyl-phenyl)-3,4-dihydro-2H-benzo[1,4]oxazine (0.018 g, 17%) as a light brown solid. LC-MS: 406 [M+H].

### Example 5

### 3-(2,6-Difluoro-phenyl)-7-(2-methyl-5-trifluoromethyl-2H-pyrazol-3-yl)-3,4-dihydro-2H-benzo[1,4]oxazine

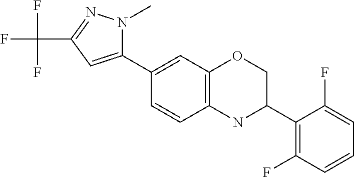 [SMILES: Cn1nc(C(F)(F)F)cc1-c1ccc2c(c1)OCC(c1c(F)cccc1F)N2]

3-(2,6-Difluoro-phenyl)-7-(2-methyl-5-trifluoromethyl-2H-pyrazol-3-yl)-3,4-dihydro-2H-benzo[1,4]oxazine: To a stirred solution of the 7-bromo-3-(2,6-difluoro-phenyl)-3,4-dihydro-2H-benzo[1,4]oxazine (0.1 g, 0.31 mmol) in 1,4-dioxane (10 mL) was added 1M K₂CO₃ (0.9 mL), Pd(PPh₃)₄(0.035 g, 0.31 mmol), SPhos (0.006 g, 0.02 mmol) and 1-methyl-3-(trifluoromethyl)-1H-pyrazol-5-ylboronic acid (0.60 g, 0.31 mmol) at rt. The mixture was then heated to 85° C. for 16 h (monitoring by TLC; ethyl acetate-hexane=1:9; Rf ˜0.2), cooled, and filtered through celite. The filtrate was concentrated under reduced pressure and purified by HPLC to give 3-(2,6-difluoro-phenyl)-7-(2-methyl-5-trifluoromethyl-2H-pyrazol-3-yl)-3,4-dihydro-2H-benzo[1,4]oxazine (0.041 g, 34%) as a light brown solid. LC-MS: 396 [M+H].

### Example 6

### 3-(2,6-Difluoro-phenyl)-7-(2-ethyl-5-trifluoromethyl-2H-pyrazol-3-yl)-3,4-dihydro-2H-benzo[1,4]oxazine

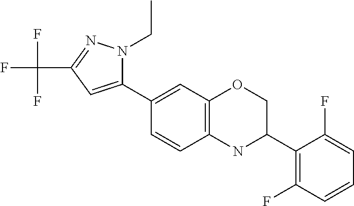 [SMILES: CCn1nc(C(F)(F)F)cc1-c1ccc2c(c1)OCC(c1c(F)cccc1F)N2]

3-(2,6-Difluoro-phenyl)-7-(2-ethyl-5-trifluoromethyl-2H-pyrazol-3-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 2-ethyl-5-trifluoromethyl-2H-pyrazol-3-yl ester in a manner identical to that described in Example 4, to give 50 mg as a yellow solid. MS: 410 [M+H].

### Example 7

### 3-(2,6-Difluoro-phenyl)-7-(2-ethyl-5-pyridin-3-yl-2H-pyrazol-3-yl)-3,4-dihydro-2H benzo[1,4]oxazine

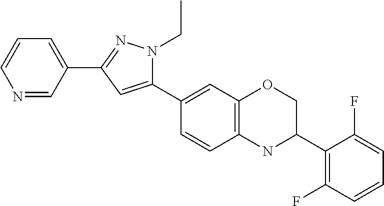 [SMILES: CCn1nc(-c2cccnc2)cc1-c1ccc2c(c1)OCC(c1c(F)cccc1F)N2]

3-(2,6-Difluoro-phenyl)-7-(2-ethyl-5-pyridin-3-yl-2H-pyrazol-3-yl)-3,4-dihydro-2H benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 2-ethyl-5-pyridin-3-yl-2H-pyrazol-3-yl ester in a manner identical to that described in Example 4, to give 38 mg as a light yellow solid. MS: 419 [M+H].

### Example 8

### 3-(2,6-Difluoro-phenyl)-7-(2-ethyl-5-pyrazin-2-yl-2H-pyrazol-3-yl)-3,4-dihydro-2H-benzo[1,4]oxazine

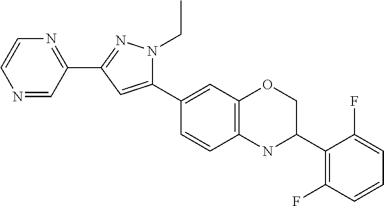 [SMILES: CCn1nc(-c2cnccn2)cc1-c1ccc2c(c1)OCC(c1c(F)cccc1F)N2]

3-(2,6-Difluoro-phenyl)-7-(2-ethyl-5-pyrazin-2-yl-2H-pyrazol-3-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 2-ethyl-5-pyrazin-2-yl-2H-pyrazol-3-yl ester in a manner identical to that described in Example 4, to give 60 mg as an off white solid. MS: 420 [M+H].

### Example 9

### 3-(2,6-Difluoro-phenyl)-7-(2-methyl-5-pyridin-3-yl-2H-[1,2,4]triazol-3-yl)-3,4-dihydro-2H-benzo[1,4]oxazine

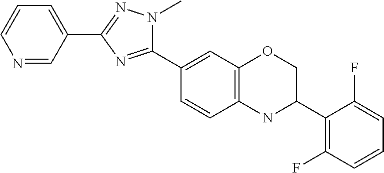 [SMILES: Cn1nc(-c2cccnc2)nc1-c1ccc2c(c1)OCC(c1c(F)cccc1F)N2]

3-(2,6-Difluoro-phenyl)-7-(2-methyl-5-pyridin-3-yl-2H-[1,2,4]triazol-3-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 3-(5-bromo-1-methyl-1H-[1,2,4]triazol-3-yl)-pyridine in a manner identical to that described in Example 4, to give 13 mg as a light yellow solid. MS: 406 [M+H].

### Example 10

### 3-(2,6-Difluoro-phenyl)-7-(2-ethyl-5-pyridin-3-yl-2H-[1,2,4]triazol-3-yl)-3,4-dihydro-2H-benzo[1,4]oxazine

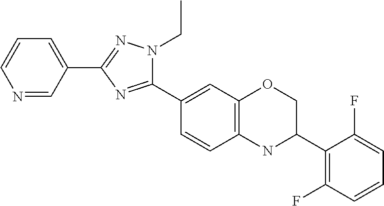 [SMILES: CCn1nc(-c2cccnc2)nc1-c1ccc2c(c1)OCC(c1c(F)cccc1F)N2]

3-(2,6-Difluoro-phenyl)-7-(2-ethyl-5-pyridin-3-yl-2H-[1,2,4]triazol-3-yl)-3,4-dihydro-2H-benzo[1,4]oxazine: To a stirred solution of 3-(2,6-difluoro-phenyl)-7-(4,4,5,5-tetramethyl-[1,3,2]dioxaborolan-2-yl)-3,4-dihydro-2H-benzo[1,4]oxazine (0.25 g, 0.67 mmol) in 1,4-dioxane (20 mL) was added 1M K₂CO₃ (2.0 mL), Pd(dppf)Cl₂, (0.057 g, 0.07 mmol), SPhos (0.014 g, 0.03 mmol) and 3-(5-bromo-1-ethyl-1H-[1,2,4]triazol-3-yl)-pyridine (0.167 g, 0.67 mmol) at rt. The mixture was then heated to 85° C. for 16 h (monitoring by TLC; ethyl acetate-hexane=1:1; Rf ˜0.2), cooled, and filtered through celite. The filtrate was concentrated under reduced pressure and purified by HPLC to give 3-(2,6-Difluoro-phenyl)-7-(2-ethyl-5-pyridin-3-yl-2H-[1,2,4]triazol-3-yl)-3,4-dihydro-2H-benzo[1,4]oxazine (0.021 g, 8%) as a brown solid. LC-MS: 420 [M+H].

### Example 11

### 3-(2,6-Difluoro-phenyl)-7-(5-methyl-2-pyridin-2-yl-thiazol-4-yl)-3,4-dihydro-2H-benzo[1,4]oxazine

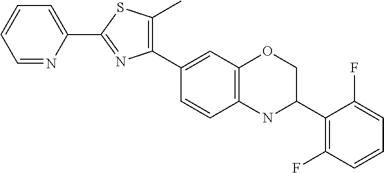 [SMILES: Cc1sc(-c2ccccn2)nc1-c1ccc2c(c1)OCC(c1c(F)cccc1F)N2]

3-(2,6-Difluoro-phenyl)-7-(5-methyl-2-pyridin-2-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-methyl-2-pyridin-2-yl-thiazol-4-yl ester in a manner identical to that described in Example 10, to give 20 mg as a light yellow solid. MS: 422 [M+H].

### Example 12

### 3-(2,6-Difluoro-phenyl)-7-(5-methyl-2-pyridin-3-yl-thiazol-4-yl)-3,4-dihydro-2H-benzo[1,4]oxazine

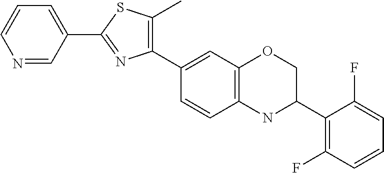 [SMILES: Cc1sc(-c2cccnc2)nc1-c1ccc2c(c1)OCC(c1c(F)cccc1F)N2]

3-(2,6-Difluoro-phenyl)-7-(5-methyl-2-pyridin-3-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-methyl-2-pyridin-3-yl-thiazol-4-yl ester in a manner identical to that described in Example 4, to give 40 mg as a light yellow solid. MS: 422 [M+H].

### Example 13

### 3-(2,6-Difluoro-phenyl)-7-(5-ethyl-2-pyridin-2-yl-thiazol-4-yl)-3,4-dihydro-2H-benzo[1,4]oxazine

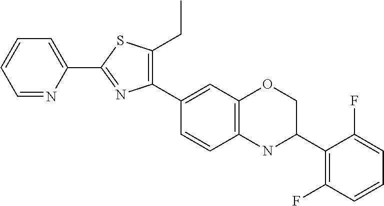 [SMILES: CCc1sc(-c2ccccn2)nc1-c1ccc2c(c1)OCC(c1c(F)cccc1F)N2]

3-(2,6-Difluoro-phenyl)-7-(5-ethyl-2-pyridin-2-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-ethyl-2-pyridin-2-yl-thiazol-4-yl ester in a manner identical to that described in Example 4, to give 19 mg as an off-white solid. MS: 436 [M+H].

### Example 14

### 3-(2,6-Difluoro-phenyl)-7-(5-ethyl-2-pyridin-3-yl-thiazol-4-yl)-3,4-dihydro-2H-benzo[1,4]oxazine

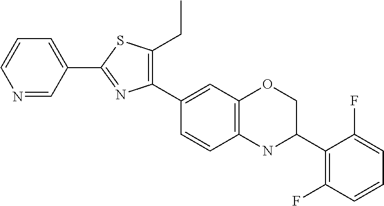 [SMILES: CCc1sc(-c2cccnc2)nc1-c1ccc2c(c1)OCC(c1c(F)cccc1F)N2]

3-(2,6-Difluoro-phenyl)-7-(5-ethyl-2-pyridin-3-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-ethyl-2-pyridin-3-yl-thiazol-4-yl ester in a manner identical to that described in Example 10, to give 40 mg as an off-white solid. MS: 436 [M+H].

### Example 15

### 3-(2,6-difluorophenyl)-7-(5-methyl-2-(pyrazin-2-yl)thiazol-4-yl)-3,4-dihydro-2H-benzo[b][1,4]oxazine

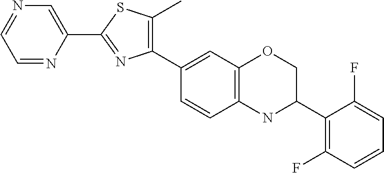 [SMILES: Cc1sc(-c2cnccn2)nc1-c1ccc2c(c1)OCC(c1c(F)cccc1F)N2]

3-(2,6-difluorophenyl)-7-(5-methyl-2-(pyrazin-2-yl)thiazol-4-yl)-3,4-dihydro-2H-benzo[b][1,4]oxazine: To a mixture of 3-(2,6-difluorophenyl)-7-(4,4,5,5-tetramethyl-1,3,2-dioxaborolan-2-yl)-3,4-dihydro-2H-benzo[b][1,4]oxazine (60 mg, 161 μmol, Eq: 1.00), 5-methyl-2-(pyrazin-2-yl)thiazol-4-yl trifluoromethanesulfonate (57.5 mg, 177 μmol, Eq: 1.1), tetrakis(triphenylphosphine)palladium (0) (18.6 mg, 16.1 μmol, Eq: 0.1) and potassium carbonate (66.7 mg, 482 μmol, Eq: 3) was added dioxane (2.86 ml) and water (715 μl). The reaction mixture was heated to 95° C. for 3 h, cooled, concentrated directly onto silica gel and chromatographed (EtOAc/Hex gradient) to give 3-(2,6-difluorophenyl)-7-(5-methyl-2-(pyrazin-2-yl)thiazol-4-yl)-3,4-dihydro-2H-benzo[b][1,4]oxazine (45 mg, 66%) as a yellow solid. MS: 423 [M+H].

### Example 16

### 3-(2,6-difluorophenyl)-7-(5-ethyl-2-(pyrazin-2-yl)thiazol-4-yl)-3,4-dihydro-2H-benzo[b][1,4]oxazine

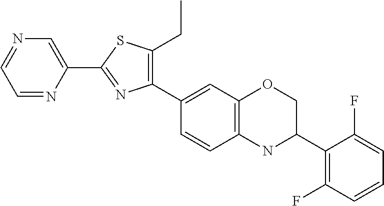 [SMILES: CCc1sc(-c2cnccn2)nc1-c1ccc2c(c1)OCC(c1c(F)cccc1F)N2]

3-(2,6-difluorophenyl)-7-(5-ethyl-2-(pyrazin-2-yl)thiazol-4-yl)-3,4-dihydro-2H-benzo[b][1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-ethyl-2-(pyrazin-2-yl)thiazol-4-yl trifluoromethanesulfonate in a manner identical to that described in Example 15, to give 31 mg as a light yellow solid. MS: 437 [M+H]

### Example 17

### 3-(2,6-Difluoro-phenyl)-7-(5-ethyl-2-pyridin-4-yl-thiazol-4-yl)-3,4-dihydro-2H-benzo[1,4]oxazine

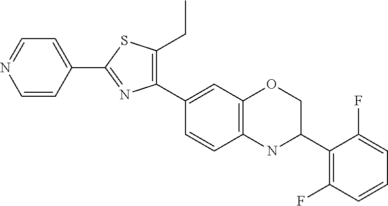 [SMILES: CCc1sc(-c2ccncc2)nc1-c1ccc2c(c1)OCC(c1c(F)cccc1F)N2]

3-(2,6-Difluoro-phenyl)-7-(5-ethyl-2-pyridin-4-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-ethyl-2-pyridin-4-yl-thiazol-4-yl ester in a manner identical to that described in Example 4, to give 60 mg as a yellow solid. MS: 436 [M+H].

### Example 18

### 3-(2,6-Difluoro-phenyl)-7-[5-ethyl-2-(2-ethyl-pyridin-4-yl)-thiazol-4-yl]-3,4-dihydro-2H-benzo[1,4]oxazine

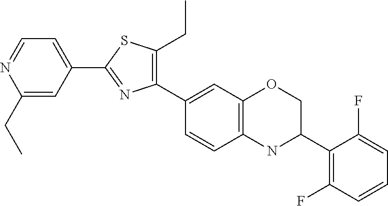 [SMILES: CCc1cc(-c2nc(-c3ccc4c(c3)OCC(c3c(F)cccc3F)N4)c(CC)s2)ccn1]

3-(2,6-Difluoro-phenyl)-7-[5-ethyl-2-(2-ethyl-pyridin-4-yl)-thiazol-4-yl]-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-ethyl-2-(2-ethyl-pyridin-4-yl)-thiazol-4-yl ester in a manner identical to that described in Example 4, to give 10 mg as a yellow solid. MS: 464 [M+H].

### Example 19

### 3-(2,6-Difluoro-phenyl)-7-(5-ethyl-2-oxazol-2-yl-thiazol-4-yl)-3,4-dihydro-2H-benzo[1,4]oxazine

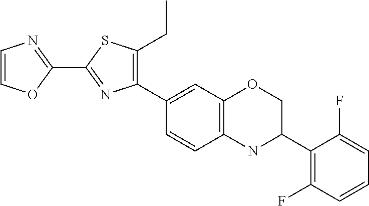 [SMILES: CCc1sc(-c2ncco2)nc1-c1ccc2c(c1)OCC(c1c(F)cccc1F)N2]

3-(2,6-Difluoro-phenyl)-7-(5-ethyl-2-oxazol-2-yl-thiazol-4-yl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-ethyl-2-oxazol-2-yl-thiazol-4-yl ester in a manner identical to that described in Example 4, to give 6 mg as an off-white solid. MS: 426 [M+H].

### Example 20

### 3-(2,6-difluorophenyl)-7-(5-ethyl-2-(5-methylisoxazol-3-yl)thiazol-4-yl)-3,4-dihydro-2H-benzo[b][1,4]oxazine

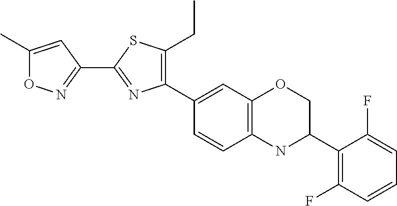 [SMILES: CCc1sc(-c2cc(C)on2)nc1-c1ccc2c(c1)OCC(c1c(F)cccc1F)N2]

3-(2,6-difluorophenyl)-7-(5-ethyl-2-(5-methylisoxazol-3-yl)thiazol-4-yl)-3,4-dihydro-2H-benzo[b][1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-ethyl-2-(5-methylisoxazol-3-yl)thiazol-4-yl trifluoromethanesulfonate in a manner identical to that described in Example 15, to give 15 mg as a yellow solid. MS: 440 [M+H]

### Example 21

### 3-(2,6-Difluoro-phenyl)-7-(6-methoxy-4-methyl-pyridin-3-yl)-3,4-dihydro-2H-benzo[1,4]oxazine

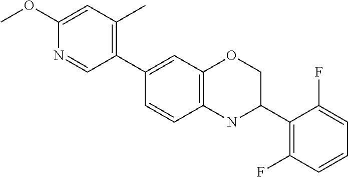 [SMILES: COc1cc(C)c(-c2ccc3c(c2)OCC(c2c(F)cccc2F)N3)cn1]

3-(2,6-Difluoro-phenyl)-7-(6-methoxy-4-methyl-pyridin-3-yl)-3,4-dihydro-2H-benzo[1,4]oxazine: Was prepared from 7-bromo-3-(2,6-difluoro-phenyl)-3,4-dihydro-2H-benzo[1,4]oxazine and 6-methoxy-4-methylpyridin-3-yl boronic acid in a manner identical to that described in Example 1, to give 51 mg as a light yellow solid. MS: 369 [M+H]

### Example 22

### 3-(2,6-difluorophenyl)-7-(6-(ethylsulfonyl)-4-methylpyridin-3-yl)-3,4-dihydro-2H-benzo[b][1,4]oxazine

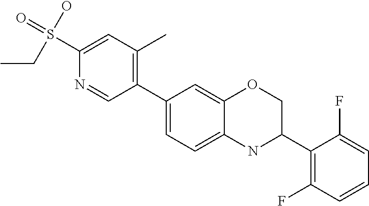

3-(2,6-difluorophenyl)-7-(6-(ethylsulfonyl)-4-methylpyridin-3-yl)-3,4-dihydro-2H-benzo[b][1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-bromo-2-(ethylsulfonyl)-4-methylpyridine in a manner identical to that described in Example 15, to give 39 mg as a yellow solid. MS: 431 [M+H]

### Example 23

### 7-(6-Chloro-4-methyl-pyridin-3-yl)-3-(2,6-difluoro-phenyl)-3,4-dihydro-2H-benzo[1,4]oxazine

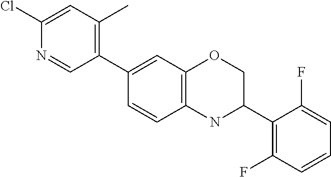 [SMILES: Cc1cc(Cl)ncc1-c1ccc2c(c1)OCC(c1c(F)cccc1F)N2]

7-(6-Chloro-4-methyl-pyridin-3-yl)-3-(2,6-difluoro-phenyl)-3,4-dihydro-2H-benzo[1,4]oxazine: Was prepared from 7-bromo-3-(2,6-difluoro-phenyl)-3,4-dihydro-2H-benzo[1,4]oxazine and 2-chloro-4-methyl-pyridine-5-boronic acid in a manner identical to that described in Example 5, to give 35 mg as a yellow solid. MS: 373 [M+H]

### Example 24

### 3-(2,6-Difluoro-phenyl)-7-(4-methyl-6-oxazol-2-yl-pyridin-3-yl)-3,4-dihydro-2H-benzo[1,4]oxazine

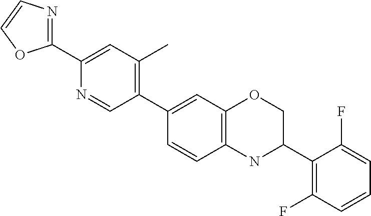 [SMILES: Cc1cc(-c2ncco2)ncc1-c1ccc2c(c1)OCC(c1c(F)cccc1F)N2]

3-(2,6-Difluoro-phenyl)-7-(4-methyl-6-oxazol-2-yl-pyridin-3-yl)-3,4-dihydro-2H-benzo[1,4]oxazine: To a solution of the 7-(6-chloro-4-methyl-pyridin-3-yl)-3-(2,6-difluoro-phenyl)-3,4-dihydro-2H-benzo[1,4]oxazine (0.035 g, 0.09 mmol) in 1,4-Dioxane (3 mL) was added Pd(dppf)Cl₂ (0.007 g, 0.01 mmol) and 2-tributylstannanyl-oxazole (0.4 ml, 0.19 mmol) at rt. The mixture was then heated to 85° C. for 16 h (monitoring by TLC; ethyl acetate-hexane=1:1; Rf ˜0.4), cooled, and filtered through celite. The filtrate was concentrated under reduced pressure and purified by HPLC to give 3-(2,6-difluoro-phenyl)-7-(4-methyl-6-oxazol-2-yl-pyridin-3-yl)-3,4-dihydro-2H-benzo[1,4]oxazine (0.012 g, 32%) as a light yellow solid. LC-MS: 406 [M+H].

### Example 25

### 5-(5-(3-(2,6-difluorophenyl)-3,4-dihydro-2H-benzo[b][1,4]oxazin-7-yl)-4-methylpyridin-2-yl)pyrimidin-2-amine

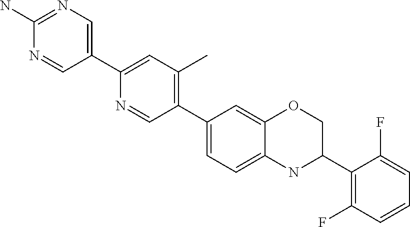 [SMILES: Cc1cc(-c2cnc(N)nc2)ncc1-c1ccc2c(c1)OCC(c1c(F)cccc1F)N2]

5-(5-(3-(2,6-difluorophenyl)-3,4-dihydro-2H-benzo[b][1,4]oxazin-7-yl)-4-methylpyridin-2-yl)pyrimidin-2-amine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-(5-bromo-4-methylpyridin-2-yl)pyrimidin-2-amine in a manner identical to that described in Example 15, to give 16 mg as a yellow solid. MS: 432 [M+H]

### Example 26

### 3-(2,6-difluorophenyl)-7-(4-methyl-6-(2-methylthiazol-5-yl)pyridin-3-yl)-3,4-dihydro-2H-benzo[b][1,4]oxazine

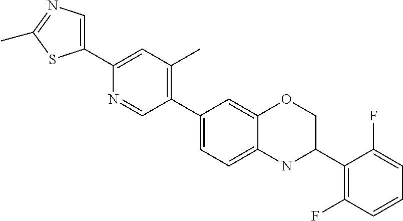 [SMILES: Cc1ncc(-c2cc(C)c(-c3ccc4c(c3)OCC(c3c(F)cccc3F)N4)cn2)s1]

3-(2,6-difluorophenyl)-7-(4-methyl-6-(2-methylthiazol-5-yl)pyridin-3-yl)-3,4-dihydro-2H-benzo[b][1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-(5-bromo-4-methylpyridin-2-yl)-2-methylthiazole in a manner identical to that described in Example 15, to give 12 mg as a yellow solid. MS: 436 [M+H]

### Example 27

### 3-(2,6-difluorophenyl)-7-(4-methyl-6-(2-methyl-2H-tetrazol-5-yl)pyridin-3-yl)-3,4-dihydro-2H-benzo[b][1,4]oxazine

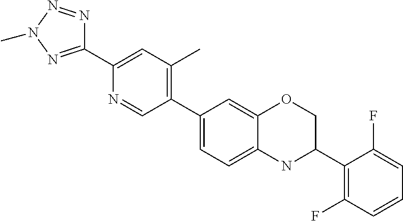 [SMILES: Cc1cc(-c2nnn(C)n2)ncc1-c1ccc2c(c1)OCC(c1c(F)cccc1F)N2]

3-(2,6-difluorophenyl)-7-(4-methyl-6-(2-methyl-2H-tetrazol-5-yl)pyridin-3-yl)-3,4-dihydro-2H-benzo[b][1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 5-bromo-4-methyl-2-(2-methyl-2H-tetrazol-5-yl)pyridine in a manner identical to that described in Example 15, to give 6 mg as a yellow solid. MS: 421 [M+H]

### Example 28

### 3-(2,6-Difluoro-phenyl)-7-(2-methyl-5-oxazol-2-yl-phenyl)-3,4-dihydro-2H-benzo[1,4]oxazine

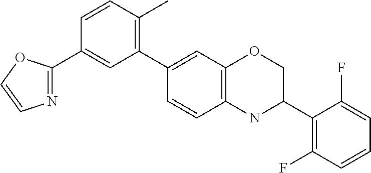 [SMILES: Cc1ccc(-c2ncco2)cc1-c1ccc2c(c1)OCC(c1c(F)cccc1F)N2]

3-(2,6-Difluoro-phenyl)-7-(2-methyl-5-oxazol-2-yl-phenyl)-3,4-dihydro-2H-benzo[1,4]oxazine: Was prepared from 3-(2,6-difluoro-phenyl)-7-(4,4,5,5-tetramethyl-[1,3,2]dioxaborolan-2-yl)-3,4-dihydro-2H-benzo[1,4]oxazine and 2-(3-Bromo-4-methyl-phenyl)-oxazole in a manner identical to that described in Example 4, to give 19 mg as a yellow solid. MS: 405 [M+H]

### Example 29

### 3-(2-Chloro-6-fluoro-phenyl)-7-(2-methyl-4-trifluoromethyl-phenyl)-3,4-dihydro-2H-benzo[1,4]oxazine

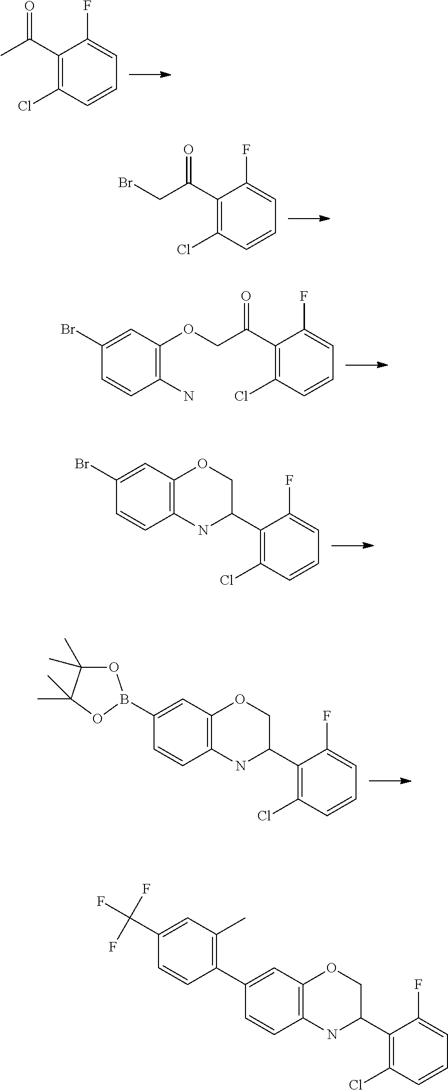

3-(2-Chloro-6-fluoro-phenyl)-7-(2-methyl-4-trifluoromethyl-phenyl)-3,4-dihydro-2H-benzo[1,4]oxazine: Was prepared starting from 1-(2-chloro-6-fluoro-phenyl)-ethanone via 3-(2-chloro-6-fluoro-phenyl)-7-(4,4,5,5-tetramethyl-[1,3,2]dioxaborolan-2-yl)-3,4-dihydro-2H-benzo[1,4]oxazine, using 1-bromo-2-methyl-4-trifluoromethyl-benzene in the final Suzuki coupling step as described in Examples 1 and 4, to give 25 mg as a yellow solid. LC-MS: 422 [M+H].

### Example 30

### 3-(2-Chloro-6-fluoro-phenyl)-7-(2-methyl-5-trifluoromethyl-phenyl)-3,4-dihydro-2H-benzo[1,4]oxazine

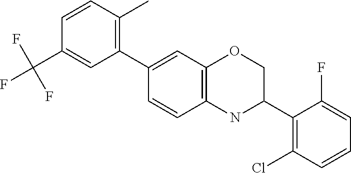 [SMILES: Cc1ccc(C(F)(F)F)cc1-c1ccc2c(c1)OCC(c1c(F)cccc1Cl)N2]

3-(2-Chloro-6-fluoro-phenyl)-7-(2-methyl-5-trifluoromethyl-phenyl)-3,4-dihydro-2H-benzo[1,4]oxazine: Was prepared from 3-(2-chloro-6-fluoro-phenyl)-7-(4,4,5,5-tetramethyl-[1,3,2]dioxaborolan-2-yl)-3,4-dihydro-2H-benzo[1,4]oxazine and 2-bromo-1-methyl-4-trifluoromethyl-benzene in a manner identical to that described in Example 29, to give 32 mg as an off-white solid. MS: 422 [M+H]

### Example 31

### 3-(2-Chloro-6-fluoro-phenyl)-7-(5-methyl-2-pyridin-2-yl-thiazol-4-yl)-3,4-dihydro-2H-benzo[1,4]oxazine

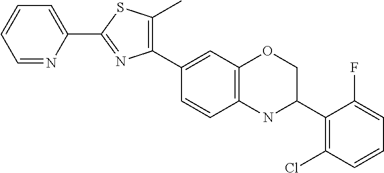 [SMILES: Cc1sc(-c2ccccn2)nc1-c1ccc2c(c1)OCC(c1c(F)cccc1Cl)N2]

3-(2-Chloro-6-fluoro-phenyl)-7-(5-methyl-2-pyridin-2-yl-thiazol-4-yl)-3,4-dihydro-2H-benzo[1,4]oxazine: Was prepared from 3-(2-chloro-6-fluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-methyl-2-pyridin-2-yl-thiazol-4-yl ester in a manner identical to that described in Example 29, to give 25 mg as a light yellow solid. MS: 438 [M+H]

### Example 32

### 3-(2-Chloro-6-fluoro-phenyl)-7-(5-methyl-2-pyridin-3-yl-thiazol-4-yl)-3,4-dihydro-2H-benzo[1,4]oxazine

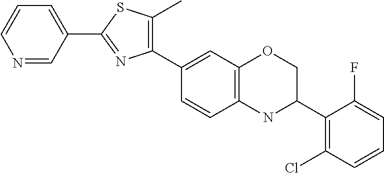 [SMILES: Cc1sc(-c2cccnc2)nc1-c1ccc2c(c1)OCC(c1c(F)cccc1Cl)N2]

3-(2-Chloro-6-fluoro-phenyl)-7-(5-methyl-2-pyridin-3-yl-thiazol-4-yl)-3,4-dihydro-2H-benzo[1,4]oxazine: Was prepared from 3-(2-chloro-6-fluoro-phenyl)-7-(4,4,5,5-tetramethyl-[1,3,2]dioxaborolan-2-yl)-3,4-dihydro-2H-benzo[1,4]oxazine and trifluoro-methanesulfonic acid 5-methyl-2-pyridin-3-yl-thiazol-4-yl ester in a manner identical to that described in Example 29, to give 40 mg as a light yellow solid. MS: 438 [M+H]

### Example 33

### 3-(2-Chloro-6-fluoro-phenyl)-7-(6-methoxy-4-methyl-pyridin-3-yl)-3,4-dihydro-2H-benzo[1,4]oxazine

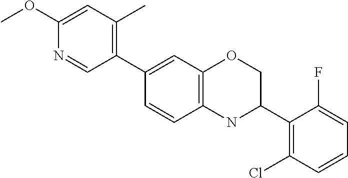 [SMILES: COc1cc(C)c(-c2ccc3c(c2)OCC(c2c(F)cccc2Cl)N3)cn1]

3-(2-Chloro-6-fluoro-phenyl)-7-(6-methoxy-4-methyl-pyridin-3-yl)-3,4-dihydro-2H-benzo[1,4]oxazine: Was prepared from 7-bromo-3-(2-chloro-6-fluoro-phenyl)-3,4-dihydro-2H-benzo[1,4]oxazine and 6-methoxy-4-methylpyridin-3-yl boronic acid in a manner identical to that described in Example 1, to give 7 mg as a light yellow solid. MS: 385 [M+H]

### Example 34

### 3-(2-chlorophenyl)-7-(1-methyl-3-(trifluoromethyl)-1H-pyrazol-5-yl)-3,4-dihydro-2H-benzo[b][1,4]oxazine

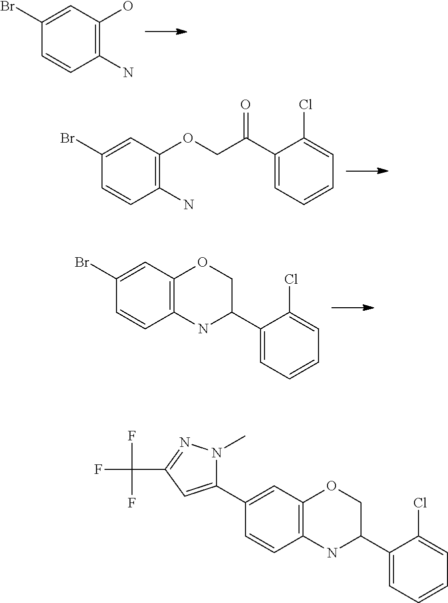 [SMILES: Nc1ccc(Br)cc1O ; Nc1ccc(Br)cc1OCC(=O)c1ccccc1Cl ; Clc1ccccc1C1COc2cc(Br)ccc2N1 ; Cn1nc(C(F)(F)F)cc1-c1ccc2c(c1)OCC(c1ccccc1Cl)N2]

2-(2-Amino-5-bromo-phenoxy)-1-(2-chloro-phenyl)-ethanone: To a solution of 2-amino-5-bromophenol (2 g, 10.6 mmol) and 2-bromo-1-(2-chlorophenyl)ethanone (2.73 g, 11.7 mmol) in CH₃CN (30 mL) was added Cs₂CO₃ (3.47 g, 10.6 mmol) at rt. The mixture was stirred at rt for 18 h, at which point it was filtered over paper and the solvent removed under reduced pressure. The resulting residue was purified by column chromatography to a give 2-(2-Amino-5-bromo-phenoxy)-1-(2-chloro-phenyl)-ethanone (1.7 g, 47%) as a yellow crystalline solid.

7-Bromo-3-(2-chloro-phenyl)-3,4-dihydro-2H-benzo[1,4]oxazine: To a solution of 2-(2-Amino-5-bromo-phenoxy)-1-(2-chloro-phenyl)-ethanone (1.45 g, 4.26 mmol) and sodium triacetoxyborohydride (2.26 g, 10.6 mmol) in DCM (60 mL) was added TFA (607 mg, 410 μL, 5.32 mmol) at rt. The mixture was stirred for 3 h (monitoring by TLC, 1:9 ethyl acetate/hexanes, Rf ˜0.4) before being quenched with aq. NaHCO3 (50 mL) and extracted with DCM (2×75 mL). The organic layers were then combined, concentrated under reduced pressure, and purified by column chromatography (5 to 10% ethyl acetate/hexanes) to give 7-bromo-3-(2-chloro-phenyl)-3,4-dihydro-2H-benzo[1,4]oxazine (1.07 g, 77%) as a thick yellow oil.

3-(2-Chlorophenyl)-7-(1-methyl-3-(trifluoromethyl)-1H-pyrazol-5-yl)-3,4-dihydro-2H-benzo[b][1,4]oxazine: To a mixture of 7-bromo-3-(2-chlorophenyl)-3,4-dihydro-2H-benzo[b][1,4]oxazine (100 mg, 308 μmol, Eq: 1.00), 1-methyl-3-(trifluoromethyl)-1H-pyrazol-5-ylboronic acid (71.7 mg, 370 μmol, Eq: 1.2), tetrakis(triphenylphosphine)palladium (0) (35.6 mg, 30.8 μmol, Eq: 0.1) and potassium carbonate (128 mg, 924 μmol, Eq: 3) was added dioxane (5.48 ml) and water (1.37 ml). The reaction mixture was then heated to 95° C. for 1 h, before being concentrated under reduced pressure, and purified by column chromatography (EtOAc/Hex gradient) to give 3-(2-chlorophenyl)-7-(1-methyl-3-(trifluoromethyl)-1H-pyrazol-5-yl)-3,4-dihydro-2H-benzo[b][1,4]oxazine (78 mg, 198 μmol, 64% yield) as a yellow solid. MS: 394.1 [M+H].

### Example 35

### 3-(2-chlorophenyl)-7-(6-methoxy-4-methylpyridin-3-yl)-3,4-dihydro-2H-benzo[b][1,4]oxazine

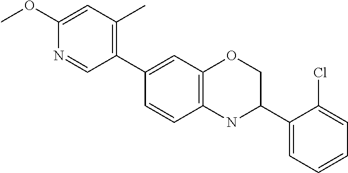 [SMILES: COc1cc(C)c(-c2ccc3c(c2)OCC(c2ccccc2Cl)N3)cn1]

3-(2-chlorophenyl)-7-(6-methoxy-4-methylpyridin-3-yl)-3,4-dihydro-2H-benzo[b][1,4]oxazine: Was prepared in a manner identical to Example 34, substituting 6-methoxy-4-methylpyridin-3-yl boronic acid in the final step to give 3-(2-chlorophenyl)-7-(6-methoxy-4-methylpyridin-3-yl)-3,4-dihydro-2H-benzo[b][1,4]oxazine (88 mg) as a yellow solid. MS: 367.1 [M+H].

### Example 36

### 3-(2-chlorophenyl)-7-(5-methyl-2-(pyridin-3-yl)thiazol-4-yl)-3,4-dihydro-2H-benzo[b][1,4]oxazine

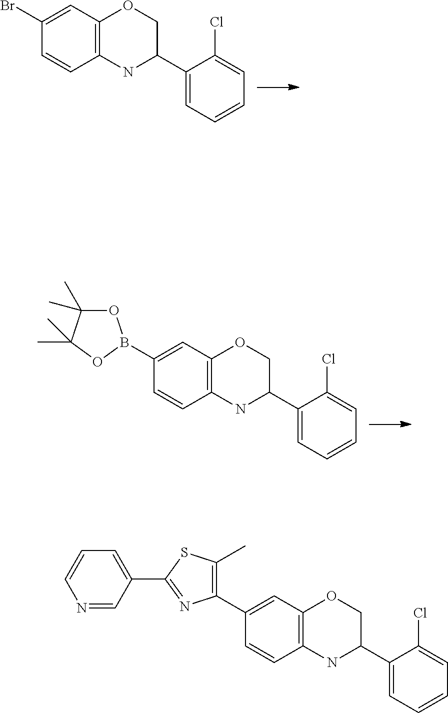 [SMILES: Clc1ccccc1C1COc2cc(Br)ccc2N1 ; CC1(C)OB(c2ccc3c(c2)OCC(c2ccccc2Cl)N3)OC1(C)C ; Cc1sc(-c2cccnc2)nc1-c1ccc2c(c1)OCC(c1ccccc1Cl)N2]

3-(2-chlorophenyl)-7-(4,4,5,5-tetramethyl-1,3,2-dioxaborolan-2-yl)-3,4-dihydro-2H-benzo[b][1,4]oxazine: To a mixture of 7-bromo-3-(2-chlorophenyl)-3,4-dihydro-2H-benzo[b][1,4]oxazine (885 mg, 2.73 mmol, Eq: 1.00), bis(pinacolato)diboron (900 mg, 3.54 mmol, Eq: 1.3), 1,1′-bis(diphenylphosphino)ferrocenedichloro palladium(II) dichloromethane complex (199 mg, 273 μmol, Eq: 0.1) and potassium acetate (803 mg, 8.18 mmol, Eq: 3) was added anhydrous dioxane (60.6 ml). The reaction mixture was heated to 110° C. for 3.5 h, at which point it was cooled and filtered over a pad of celite. The resulting filtrate was diluted with EtOAc, washed with water and brine, dried over magnesium sulfate, concentrated under reduced pressure, and purified by column chromatography (EtOAc/Hex gradient) to give 3-(2-chlorophenyl)-7-(4,4,5,5-tetramethyl-1,3,2-dioxaborolan-2-yl)-3,4-dihydro-2H-benzo[b][1,4]oxazine (433 mg, 1.17 mmol, 42.7% yield) as a light brown gum.

3-(2-chlorophenyl)-7-(5-methyl-2-(pyridin-3-yl)thiazol-4-yl)-3,4-dihydro-2H-benzo[b][1,4]oxazine: To a mixture of 3-(2-chlorophenyl)-7-(4,4,5,5-tetramethyl-1,3,2-dioxaborolan-2-yl)-3,4-dihydro-2H-benzo[b][1,4]oxazine (60 mg, 161 μmol, Eq: 1.00), 5-methyl-2-(pyridin-3-yl)thiazol-4-yl trifluoromethanesulfonate (57.6 mg, 178 μmol, Eq: 1.1), tetrakis(triphenylphosphine)palladium (0) (18.7 mg, 16.1 μmol, Eq: 0.1) and potassium carbonate (66.9 mg, 484 μmol, Eq: 3) was added dioxane (2.87 ml) and water (717 μl). The reaction mixture was heated to 95° C. for 2.5 h, before being concentrated under reduced pressure, and purified by column chromatography (15-45% EtOAc/Hex gradient) providing a solid that was further purified by trituration with diethyl ether to give 3-(2-chlorophenyl)-7-(5-methyl-2-(pyridin-3-yl)thiazol-4-yl)-3,4-dihydro-2H-benzo[b][1,4]oxazine (36 mg, 85.7 μmol, 53% yield) as a light orange solid. MS: 420.1 [M+H].

### Example 37

### 3-(2-chlorophenyl)-7-(5-ethyl-2-(pyrazin-2-yl)thiazol-4-yl)-3,4-dihydro-2H-benzo[b][1,4]oxazine

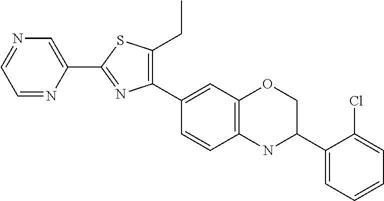 [SMILES: CCc1sc(-c2cnccn2)nc1-c1ccc2c(c1)OCC(c1ccccc1Cl)N2]

3-(2-chlorophenyl)-7-(5-ethyl-2-(pyrazin-2-yl)thiazol-4-yl)-3,4-dihydro-2H-benzo[b][1,4]oxazine: Was prepared in a manner identical to Example 36, substituting 5-ethyl-2-(pyrazin-2-yl)thiazol-4-yl trifluoromethanesulfonate in the final step to give 3-(2-chlorophenyl)-7-(5-ethyl-2-(pyrazin-2-yl)thiazol-4-yl)-3,4-dihydro-2H-benzo[b][1,4]oxazine (13 mg) as an off-white solid. MS: 435.2 [M+H].

### Example 38

Assays

### Jurkat IL-2 Production Assay

Cell:

Jurkat cell (ATCC) was grown in RPMI 1640 with 10% FBS and 1% penicillin/streptomycin. The cell density was kept at 1.2˜1.8×10⁶/mL in culture flask before seeding into culture plate, and the cell density in the plate was 0.5×10⁶/2004/well.

Culture Media:

RPMI 1640 with 1% FBS or 30% FBS for high serum assay.

Test Compound:

Serial dilution was done in 100% DMSO, and intermediate dilution was done with RPMI 1640 medium with 1% FBS. The DMSO final concentration in culture well was 0.25%.

Stimulant:

PHA (Sigma#L9017-10MG) was used for the assay with 1% FBS in culture medium, and added after 10 minutes exposure of cell to compound/DMSO. The PHA final concentration in culture well was 5 μg/mL. PMA (Sigma# P-8139 5MG)/Ionomycin (Sigma# I0634-5MG) was used for the assay with 30% FBS in culture medium, and added at same time point as the 1% FBS culture assay. The final concentration of PMA was 50 ng/mL, and Ionomycin final concentration was 500 ng/mL.

Incubation:

At 37° C. with 5% CO₂ and 95% humidity for 18 h˜20 h.

IC50:

IC50 was calculated with the data analysis software XLfit4, General Pharmacology model 251.

### Human Whole Blood Assay

Inhibition of T cell IL-2 production CRAC compounds were tested in human whole blood from normal donors. 200 μl of human whole blood was aliquoted into each well of a round bottom 96 well plate. Concentration-response curves were generated for test compounds and CsA (as controls) by performing serial dilutions of the compounds and incubating each concentration in a single well for 30 min (experiment was performed in triplicate). Following incubation, the blood was stimulated with 10 μM Thapsigargin (SIGMA) overnight at 37° C. IL-2 concentrations in the plasma were determined by AlphaLISA IL-2 assay (Perkin Elmer). Data analysis was performed using GraphPad PRISMS.

### [3H]Thymidine Incorporation (MLR)

Freshly isolated PBMC from healthy donors were either used as responders, where the cells were pre-treated with various concentrations of inhibitors (as indicated in the figure legends), or stimulators where the cells were pre-treated with 0.05 mg/ml Mitomycin C for 20 min. Stimulator cells were washed twice with RPMI 1640 and mixed with responder PBMCs at a 1:1 ratio, totaling 2×105 cells/well in quadruplicate in a 96-well U-bottom tissue culture plates (Falcon). Cells were cultured in complete RPMI 1640 (supplemented with 10% FBS, 1 mM sodium pyruvate, and 100 IU/ml penicillin-streptomycin, 0.01M HEPES and 55 μM β-ME [Invitrogen]) for 4 days at 37° C. in 5% CO2. Cultures were pulsed with 0.5 μCi [3H]-thymidine (PerkinElmer) during the last 6 hrs of culture. Cells were harvested on filter plates using a 96-well plate harvester (PerkinElmer), and the incorporation of [3H]-thymidine was measured as cpm in a Topcount Microplate scintillation counter (PerkinElmer). Stimulator cells alone or responder cells alone were set up in parallel as controls.

Using the above procedures, IC₅₀, HWB and MLR values for certain compounds of the invention were determined and are shown in Table 1:

TABLE 1

| Example | Ic50 | HWB | MLR |
|---|---|---|---|
| Number | (μM) | (μM) | (μM) |
| 1 | 0.12 | | |
| 2 | 0.08 | 3.16 | |
| 3 | 0.35 | | |
| 4 | 0.10 | 3.22 | |
| 5 | 0.10 | 0.91 | |
| 6 | 0.02 | 0.51 | 0.11 |
| 7 | 0.07 | 0.32 | 0.33 |
| 8 | 0.11 | | |
| 9 | 0.31 | | |
| 10 | 0.09 | 0.51 | |
| 11 | 0.12 | 1.82 | |
| 12 | 0.03 | 0.43 | 0.19 |
| 13 | | 1.94 | |
| 14 | 0.03 | 0.52 | 0.10 |
| 15 | 0.06 | | |
| 16 | 0.02 | 0.87 | 0.18 |
| 17 | 0.02 | 0.32 | 0.44 |
| 18 | | 6.15 | |
| 19 | | 0.65 | |
| 20 | 0.03 | 0.36 | 0.43 |
| 21 | 0.28 | | |
| 22 | 0.09 | | |
| 23 | 0.32 | | |
| 24 | 0.34 | | |
| 25 | 0.66 | | |
| 26 | 0.21 | | |
| 27 | 0.67 | | |
| 28 | | 1.00 | |
| 29 | 0.16 | 5.33 | |
| 30 | 0.22 | | |
| 31 | 0.14 | 3.55 | |
| 32 | 0.05 | 0.58 | |
| 33 | 0.38 | | |
| 34 | 0.35 | | |
| 35 | 0.73 | | |
| 36 | 0.09 | | |
| 37 | 0.07 | | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed is:

1. A compound of formula (I): 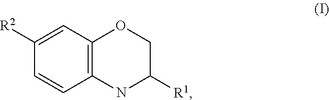
wherein:
R¹ is phenyl, unsubstituted or mono- or bi-substituted independently with halogen; and
R² is:
phenyl, unsubstituted or mono- or bi-substituted independently with lower alkyl, halogen, halo-lower alkyl, alkoxy, unsubstituted five-membered heteroaryl ring or five-membered heteroaryl ring substituted with lower alkyl;
pyridine, unsubstituted or mono- or bi-substituted independently with lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety; or
a five-membered heteroaryl ring, unsubstituted or mono- or bi-substituted independently with lower alkyl, halogen, halo-lower alkyl, alkoxy, unsubstituted five-membered heteroaryl ring, five-membered heteroaryl ring substituted with lower alkyl, unsubstituted six-membered heteroaryl ring or six-membered heteroaryl ring substituted with lower alkyl;
or a pharmaceutically acceptable salt thereof.

2. The compound according to claim 1, wherein R¹ is phenyl mono- or bi-substituted independently with F or Cl.

3. The compound according to claim 1, wherein R¹ is difluoro-phenyl.

4. The compound according to claim 1, wherein R¹ is chloro-fluoro-phenyl.

5. The compound according to claim 1, wherein R² is phenyl, unsubstituted or mono- or bi-substituted independently with lower alkyl, halogen, halo-lower alkyl, alkoxy, unsubstituted five-membered heteroaryl ring or five-membered heteroaryl ring substituted with lower alkyl.

6. The compound according to claim 1, wherein R² is phenyl substituted with —CF₃ and —CH₃.

7. The compound according to claim 1, wherein R² is pyridine, unsubstituted or mono- or bi-substituted independently with lower alkyl, halogen, halo-lower alkyl, alkoxy, SO₂CH₂CH₃, unsubstituted five-membered heteroaryl ring, five-membered heteroaryl ring substituted with lower alkyl, unsubstituted six-membered heteroaryl ring or six-membered heteroaryl ring substituted with an amino moiety.

8. The compound according to claim 1, wherein R is pyridine, unsubstituted or mono- or bi-substituted independently with —CH₃, —OCH₃, —SO₂CH₂CH₃, chlorine, oxazole, methyl-pyrimidine-amine, methyl-thiazole or methyl-tetrazole.

9. The compound according to claim 1, wherein R² is a five-membered heteroaryl ring, unsubstituted or mono- or bi-substituted independently with lower alkyl, halogen, halo-lower alkyl, alkoxy, unsubstituted five-membered heteroaryl ring, five-membered heteroaryl ring substituted with lower alkyl, unsubstituted six-membered heteroaryl ring or six-membered heteroaryl ring substituted with lower alkyl.

10. The compound according to claim 1, wherein R² is pyrazole, triazole or thiazole, mono- or bi-substituted independently with —CF3, methyl, ethyl, pyridine, pyrazine methyl-pyridine, oxazole or methyl-oxazole.

11. The compound according to claim 1, wherein said compound is:
3-(2,6-Difluoro-phenyl)-7-(2-methyl-4-trifluoromethyl-phenyl)-3,4-dihydro-2H-benzo[1,4]oxazine;
(R)-3-(2,6-Difluoro-phenyl)-7-(2-methyl-4-trifluoromethyl-phenyl)-3,4-dihydro-2H-benzo[1,4]oxazine or (S)-3-(2,6-Difluoro-phenyl)-7-(2-methyl-4-trifluoromethyl-phenyl)-3,4-dihydro-2H-benzo[1,4]oxazine;
(S)-3-(2,6-Difluoro-phenyl)-7-(2-methyl-4-trifluoromethyl-phenyl)-3,4-dihydro-2H-benzo[1,4]oxazine or (R)-3-(2,6-Difluoro-phenyl)-7-(2-methyl-4-trifluoromethyl-phenyl)-3,4-dihydro-2H-benzo[1,4]oxazine;
3-(2,6-difluoro-phenyl)-7-(2-methyl-5-trifluoromethyl-phenyl)-3,4-dihydro-2H-benzo[1,4]oxazine;
3-(2,6-Difluoro-phenyl)-7-(2-methyl-5-trifluoromethyl-2H-pyrazol-3-yl)-3,4-dihydro-2H-benzo[1,4]oxazine;
3-(2,6-Difluoro-phenyl)-7-(2-ethyl-5-trifluoromethyl-2H-pyrazol-3-yl)-3,4-dihydro-2H-benzo[1,4]oxazine;
3-(2,6-Difluoro-phenyl)-7-(2-ethyl-5-pyridin-3-yl-2H-pyrazol-3-yl)-3,4-dihydro-2H benzo[1,4]oxazine;
3-(2,6-Difluoro-phenyl)-7-(2-ethyl-5-pyrazin-2-yl-2H-pyrazol-3-yl)-3,4-dihydro-2H-benzo[1,4]oxazine;
3-(2,6-Difluoro-phenyl)-7-(2-methyl-5-pyridin-3-yl-2H-[1,2,4]triazol-3-yl)-3,4-dihydro-2H-benzo[1,4]oxazine;
3-(2,6-Difluoro-phenyl)-7-(2-ethyl-5-pyridin-3-yl-2H-[1,2,4]triazol-3-yl)-3,4-dihydro-2H-benzo[1,4]oxazine;
3-(2,6-Difluoro-phenyl)-7-(5-methyl-2-pyridin-2-yl-thiazol-4-yl)-3,4-dihydro-2H-benzo[1,4]oxazine;
3-(2,6-Difluoro-phenyl)-7-(5-methyl-2-pyridin-3-yl-thiazol-4-yl)-3,4-dihydro-2H-benzo[1,4]oxazine;
3-(2,6-Difluoro-phenyl)-7-(5-ethyl-2-pyridin-2-yl-thiazol-4-yl)-3,4-dihydro-2H-benzo[1,4]oxazine;
3-(2,6-Difluoro-phenyl)-7-(5-ethyl-2-pyridin-3-yl-thiazol-4-yl)-3,4-dihydro-2H-benzo[1,4]oxazine;
3-(2,6-difluorophenyl)-7-(5-methyl-2-(pyrazin-2-yl)thiazol-4-yl)-3,4-dihydro-2H-benzo[b][1,4]oxazine;
3-(2,6-difluorophenyl)-7-(5-ethyl-2-(pyrazin-2-yl)thiazol-4-yl)-3,4-dihydro-2H-benzo[b][1,4]oxazine;
3-(2,6-Difluoro-phenyl)-7-(5-ethyl-2-pyridin-4-yl-thiazol-4-yl)-3,4-dihydro-2H-benzo[1,4]oxazine;
3-(2,6-Difluoro-phenyl)-7-[5-ethyl-2-(2-ethyl-pyridin-4-yl)-thiazol-4-yl]-3,4-dihydro-2H-benzo[1,4]oxazine;
3-(2,6-Difluoro-phenyl)-7-(5-ethyl-2-oxazol-2-yl-thiazol-4-yl)-3,4-dihydro-2H-benzo[1,4]oxazine;
3-(2,6-difluorophenyl)-7-(5-ethyl-2-(5-methylisoxazol-3-yl)thiazol-4-yl)-3,4-dihydro-2H-benzo[b][1,4]oxazine;
3-(2,6-Difluoro-phenyl)-7-(6-methoxy-4-methyl-pyridin-3-yl)-3,4-dihydro-2H-benzo[1,4]oxazine;
3-(2,6-difluorophenyl)-7-(6-(ethylsulfonyl)-4-methylpyridin-3-yl)-3,4-dihydro-2H-benzo[b][1,4]oxazine;
7-(6-Chloro-4-methyl-pyridin-3-yl)-3-(2,6-difluoro-phenyl)-3,4-dihydro-2H-benzo[1,4]oxazine;
3-(2,6-Difluoro-phenyl)-7-(4-methyl-6-oxazol-2-yl-pyridin-3-yl)-3,4-dihydro-2H-benzo[1,4]oxazine;
5-(5-(3-(2,6-difluorophenyl)-3,4-dihydro-2H-benzo[b][1,4]oxazin-7-yl)-4-methylpyridin-2-yl)pyrimidin-2-amine;
3-(2,6-difluorophenyl)-7-(4-methyl-6-(2-methylthiazol-5-yl)pyridin-3-yl)-3,4-dihydro-2H-benzo[b][1,4]oxazine;
3-(2,6-difluorophenyl)-7-(4-methyl-6-(2-methyl-2H-tetrazol-5-yl)pyridin-3-yl)-3,4-dihydro-2H-benzo[b][1,4]oxazine;
3-(2,6-Difluoro-phenyl)-7-(2-methyl-5-oxazol-2-yl-phenyl)-3,4-dihydro-2H-benzo[1,4]oxazine;
3-(2-Chloro-6-fluoro-phenyl)-7-(2-methyl-4-trifluoromethyl-phenyl)-3,4-dihydro-2H-benzo[1,4]oxazine;
3-(2-Chloro-6-fluoro-phenyl)-7-(2-methyl-5-trifluoromethyl-phenyl)-3,4-dihydro-2H-benzo[1,4]oxazine;
3-(2-Chloro-6-fluoro-phenyl)-7-(5-methyl-2-pyridin-2-yl-thiazol-4-yl)-3,4-dihydro-2H-benzo[1,4]oxazine;
3-(2-Chloro-6-fluoro-phenyl)-7-(5-methyl-2-pyridin-3-yl-thiazol-4-yl)-3,4-dihydro-2H-benzo[1,4]oxazine;
3-(2-Chloro-6-fluoro-phenyl)-7-(6-methoxy-4-methyl-pyridin-3-yl)-3,4-dihydro-2H-benzo[1,4]oxazine;
3-(2-chlorophenyl)-7-(1-methyl-3-(trifluoromethyl)-1H-pyrazol-5-yl)-3,4-dihydro-2H-benzo[b][1,4]oxazine;
3-(2-chlorophenyl)-7-(6-methoxy-4-methylpyridin-3-yl)-3,4-dihydro-2H-benzo[b][1,4]oxazine;
3-(2-chlorophenyl)-7-(5-methyl-2-(pyridin-3-yl)thiazol-4-yl)-3,4-dihydro-2H-benzo[b][1,4]oxazine or
3-(2-chlorophenyl)-7-(5-ethyl-2-(pyrazin-2-yl)thiazol-4-yl)-3,4-dihydro-2H-benzo[b][1,4]oxazine.

12. A pharmaceutical composition, comprising a therapeutically effective amount of a compound according to claim 1 and a therapeutically inert carrier.

13. A method for the treatment or prophylaxis of arthritis or a respiratory disorder, which method comprises the step of administering a therapeutically effective amount of a compound according to claim 1 to a patient in need thereof.

14. The method according to claim 14, wherein said respiratory disorder is chronic obstructive pulmonary disorder (COPD), asthma or bronchospasm.
